(19) United States
(12) Patent Application Publication
(10) Pub. No.: US 2010/0249700 A1
(43) Pub. Date: Sep. 30, 2010

(54) SURGICAL INSTRUMENTS FOR IN VIVO ASSEMBLY

(75) Inventor: James T. Spivey, Cincinnati, OH (US)

Correspondence Address: K&L Gates LLP, 210 SIXTH AVENUE, PITTSBURGH, PA, 15222-2613, US

(73) Assignee: Ethicon Endo-Surgery, Inc., Cincinnati, OH (US)

(21) Appl. No.: 12/413,479

(22) Filed: Mar. 27, 2009

(51) Int. Cl. A61M 25/00; A61M 29/00

(52) U.S. Cl. 604/96.01; 604/264

(57) ABSTRACT

A method of assembling a surgical instrument inside a patient and a device likewise configured to be assembled in vivo through a body wall of the patient is provided. In at least one embodiment, the method includes delivering an end effector to a body cavity of the patient, inserting a shaft into the body cavity, and connecting the shaft to the end effector inside the body cavity. In such embodiments, the end effector can be operably engaged with a flexible member, wherein pulling the flexible member can cause the end effector to move toward the shaft such that the end effector connects to the shaft.

### BACKGROUND

The present invention is related generally to medical devices and more particularly to devices and methods useful in endoscopic procedures.

Access to the abdominal cavity may, from time to time, be required for diagnostic and therapeutic endeavors for a variety of medical and surgical diseases. Historically, abdominal access has required a formal laparotomy to provide adequate exposure. Such procedures, which require large incisions to be made in the abdomen, are not particularly well-suited for patients that may have extensive abdominal scarring from previous procedures, those persons who are morbidly obese, those individuals with abdominal wall infection, and those patients with diminished abdominal wall integrity, such as patients with burns and skin grafting. Other patients simply do not want to have a large scar if it can be avoided.

Minimally invasive procedures are desirable because such procedures can reduce pain and provide relatively quick recovery times as compared with conventional open medical procedures. Many minimally invasive procedures are performed with an endoscope (including, without limitation, laparoscopes). Such procedures permit a physician to position, manipulate, and view medical instruments and accessories inside the patient through a small access opening in the patient's body. Laparoscopy is a term used to describe such an “endosurgical” approach using an endoscope (often a rigid laparoscope). In this type of procedure, accessory devices are often inserted into a patient through trocars placed through the body wall. Trocars must typically pass through several layers of overlapping tissue/muscle before reaching the abdominal cavity.

Still less invasive treatments include those that are performed through insertion of an endoscope through a natural body orifice to a treatment region. Examples of this approach include, but are not limited to, cholecystectomy, appendectomy, cystoscopy, hysteroscopy, esophagogastroduodenoscopy, and colonoscopy. Many of these procedures employ the use of a flexible endoscope during the procedure. Flexible endoscopes often have a flexible, steerable articulating section near the distal end that can be controlled by the user by utilizing controls at the proximal end. Minimally invasive therapeutic procedures to treat diseased tissue by introducing medical instruments to a tissue treatment region through a natural opening of the patient are known as Natural Orifice Translumenal Endoscopic Surgery (NOTES)™.

Some flexible endoscopes are relatively small (about 1 mm to 3 mm in diameter), and may have no integral accessory channel (also called biopsy channels or working channels). Other flexible endoscopes, including gastroscopes and colonoscopes, have integral working channels having a diameter of about 2.0 mm to 3.5 mm for the purpose of introducing and removing medical devices and other accessory devices to perform diagnosis or therapy within the patient. As a result, the accessory devices used by a physician can be limited in size by the diameter of the accessory channel of the scope used. Additionally, the physician may be limited to a single accessory device when using the standard endoscope having one working channel.

Certain specialized endoscopes are available, such as large working channel endoscopes having a working channel of about 5 mm in diameter, which can be used to pass relatively large accessories, or to provide capability to suction large blood clots. Other specialized endoscopes include those having two or more working channels.

The above mentioned minimally invasive surgical procedures have changed some of the major open surgical procedures such as gall bladder removal, or a cholecystectomy, to simple outpatient surgery. Consequently, the patient's recovery time has changed from weeks to days. These types of surgeries are often used for repairing defects or for the removal of diseased tissue or organs from areas of the body such as the abdominal cavity.

Further, as the range of therapeutic endolumenal and transgastric treatments available to gastroenterologists and surgeons expands, the tools used to perform such procedures are becoming more complex. Increasingly sophisticated maneuvers demand greater functionality within the limited space offered by the gastrointestinal tract. Consequently, the size of surgical end effectors developed to achieve this functionality will increase, preventing operation through a working channel of an endoscope, which is the traditional approach to endoscopic procedures. Instruments have been developed that provide control of tools extending tangential to an endoscope. For example, accessory channels that run along side an endoscope have been developed with steering mechanisms at the distal end for effecting movement of a tool inserted therethrough.

The foregoing discussion is intended only to illustrate the present field of the invention and should not be taken as a disavowal of claim scope.

### SUMMARY

In various embodiments, a surgical kit can be configured to be assembled by a user to form a surgical instrument while various parts of the kit and/or the surgical instrument are at least partially inside of a patient. In at least one embodiment, the surgical kit can comprise an end effector configured to be delivered into a body cavity of a patient, a flexible member extending from the end effector, and a cannula. In these embodiments, the cannula can include a first end configured to be inserted into the body cavity, a second end, and an aperture that is sized and configured to receive at least a portion of the flexible member. Further, in these embodiments, the first end can include a connector portion configured to be releasably attached to the end effector.

In at least one embodiment, a surgical instrument can comprise an end effector configured to be delivered to a body cavity of a patient through a natural opening in the patient, an elongate flexible member extending from the end effector, a cannula, and an actuation shaft comprising an attachment portion. In these embodiments, the cannula can include a first end configured to be inserted into the body cavity through a second opening in the patient, a second end, and an aperture that is sized and configured to receive at least a portion of the flexible member. Further, in these embodiments, the first end can include a connector portion configured to be releasably attached to the end effector. Additionally, in these embodiments, the attachment portion of the actuation shaft can be configured to be releasably attached to the end effector such that operation of the actuation shaft can operate the end effector.

In at least one embodiment, a method of assembling a surgical instrument inside a patient is provided that can include the steps of delivering an end effector operably engaged with a flexible member to a body cavity of the patient, inserting a shaft into the body cavity, pulling the flexible member relative to the shaft such that the end effector moves relative to the shaft, and connecting the shaft to the end effector inside the body cavity.

In at least one embodiment, a method of assembling a surgical instrument inside a patient is provided that can include the steps of delivering an end effector to a body cavity of the patient, inserting a shaft into the body cavity, and connecting the shaft to the end effector inside the body cavity.

In at least one embodiment, a method of assembling a patient inside a patient is provided that can include the steps of delivering an end effector to a body cavity of the patient, inserting a shaft into the body cavity, and connecting the shaft to the end effector inside the body cavity. In these embodiments, the end effector can be operably engaged with a flexible member. Pulling the flexible member may cause the end effector and the shaft to move relatively toward each other such that the shaft connects to the end effector.

In at least one embodiment, a method of assembling a surgical instrument inside a patient is provided that can include the steps of delivering an end effector operably engaged with a flexible member to a body cavity of the patient, puncturing a body wall of the body cavity to create a port, introducing a capturing device into the body cavity through the port, capturing the flexible member with the capturing device, pulling the capturing device through the port, inserting a shaft into the body cavity through the port, and connecting the shaft to the end effector inside the body cavity.

In at least one embodiment, a method of assembling a surgical instrument is provided that can include the steps of passing a flexible member operably engaged with an end effector through a cannula, pulling the flexible member to connect the end effector to the cannula, inserting the flexible member through an actuation shaft, translating the actuation shaft along the flexible member, through the cannula, and into the end effector, coupling the actuation shaft to an actuator of the end effector, and attaching the actuation shaft and the cannula to a handle, thereby forming the surgical instrument.

In at least one embodiment, a method of assembling a surgical instrument is provided that can include the steps of delivering an end effector operably engaged with a flexible member to a body cavity of the patient, passing the flexible member through a body wall of the patient, translating a shaft along the flexible member through the body wall and into the body cavity, and connecting the end effector to the shaft inside the body cavity.

In at least one embodiment, a method of retracting a body wall inside a patient is provided that can include the steps of delivering an expandable bolster to a body cavity of the patient, passing a member operably engaged with the expandable bolster through a body wall of the patient, expanding the expandable bolster to create an expanded bolster, and pulling the expanded bolster to retract the body wall of the patient.

This Summary is intended to briefly outline certain embodiments of the subject application. It should be understood that the subject application is not limited to the embodiments disclosed in this Summary, and is intended to cover modifications that are within its spirit and scope, as defined by the claims. It should be further understood that this Summary should not be read or construed in a manner that will act to narrow the scope of the claims.

### BRIEF DESCRIPTION OF THE FIGURES

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1A is a diagrammatical view illustrating a non-limiting embodiment of an endoscope inserted into an overtube and through a patient's mouth and esophagus to perform a surgical activity such as to remove the patient's gall bladder, or perform a cholecystectomy, for example.

FIG. 1B is a diagrammatical view illustrating a non-limiting embodiment of an end effector that has been delivered to the body cavity of the patient of FIG. 1A, and a cannula that has been inserted through the patient's abdominal wall.

FIG. 1C is a diagrammatical view illustrating a non-limiting embodiment of the end effector after it has been connected to the cannula inside the body cavity of the patient of FIG. 1A.

FIG. 1D is a diagrammatical view illustrating a non-limiting embodiment of the end effector after it has been connected to both the cannula and an actuation shaft inside the body cavity of the patient of FIG. 1A.

FIG. 1E is a diagrammatical view illustrating a non-limiting embodiment of a surgical instrument that has been assembled while at least partially inside the body cavity of the patient of FIG. 1A; the assembled surgical instrument includes the end effector and cannula of FIG. 1B, the actuation shaft of FIG. 1D, and a handle.

FIG. 2 is a partial perspective view of the distal portion of the endoscope inserted through the overtube of FIG. 1A.

FIG. 3 is perspective view of the surgical instrument of FIG. 1E.

FIG. 4 is a perspective view of the cannula of the surgical instrument of FIG. 1E.

FIG. 5 is a perspective view of the actuation shaft of the surgical instrument of FIG. 1E.

FIG. 6A is a perspective view of the handle of the surgical instrument of FIG. 1E.

FIG. 6B is a back view of the handle of the surgical instrument of FIG. 1E.

FIG. 6C is an exploded view of the handle of the surgical instrument of FIG. 1E.

FIG. 6D is another exploded view of the handle of the surgical instrument of FIG. 1E.

FIG. 7A is a perspective view of the end effector of the surgical instrument of FIG. 1E.

FIG. 7B is an exploded view of the end effector of the surgical instrument of FIG. 1E.

FIG. 8A is a side cross-sectional view of the end effector connected to the cannula of FIG. 1C.

FIG. 8B is a side cross-sectional view of the end effector connected to the cannula and to the actuation shaft of FIG. 1D.

FIG. 8C is a side cross-sectional view of the surgical instrument of FIG. 1E with the end effector in an open, unactuated configuration.

FIG. 8D is a side cross-sectional view of the surgical instrument of FIG. 1E with the end effector in a closed, actuated configuration.

FIG. 9 is a partial perspective cross-sectional view of the surgical instrument of FIG. 1E showing a ratchet mechanism that is formed between parts of the actuation shaft and the handle.

FIG. 10 is a perspective view of another non-limiting embodiment of a surgical instrument.

FIGS. 11-35 illustrate an in vivo method of assembling the surgical instrument of FIG. 10.

FIG. 36 shows various non-limiting end effectors for use in a surgical instrument, which include an expandable bolster, a 5 mm Maryland-style dissector, a 10 mm Babcock-style grasper, and a 5 mm grasper.

FIG. 37A is a cross-sectional view of the expandable bolster of FIG. 36 in a collapsed, unactuated configuration.

FIG. 37B is a cross-sectional view of the expandable bolster of FIG. 36 in an expanded, actuated configuration.

FIG. 38A is a perspective view of the expandable bolster of FIG. 36 in an expanded, actuated configuration and being pulled against a body wall to increase working space inside a patient's body.

FIG. 38B is a cross-sectional view of the expandable bolster of FIG. 36 in an expanded, actuated configuration and being pulled against a body wall to create space inside a patient's body.

FIG. 39 is a diagrammatical view illustrating a non-limiting embodiment of an end effector comprising a needle knife after it has been connected to a cannula inside the body cavity of the patient of FIG. 1A.

FIG. 40 is a diagrammatical view illustrating a non-limiting embodiment of an end effector comprising a sphincterotome after it has been connected to a cannula inside the body cavity of the patient of FIG. 1A.

Corresponding reference characters indicate like or corresponding parts throughout the several views. The various illustrated embodiments have been chosen for the convenience of the reader and not to limit the scope of the appended claims.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the various embodiments of the present invention is defined solely by the claims. The features illustrated or described in connection with one exemplary embodiment may be combined with the features of other embodiments. Such modifications and variations are intended to be included within the scope of the present invention.

In the following description, like reference characters designate like or corresponding parts throughout the several views. Also in the following description, it is to be understood that terms such as “forward,” “rearward,” “front,” “back,” “right,” “left,” “upwardly,” “downwardly,” and the like are words of convenience and are not to be construed as limiting terms. The description below is for the purpose of describing various embodiments of the invention and is not intended to limit the invention thereto.

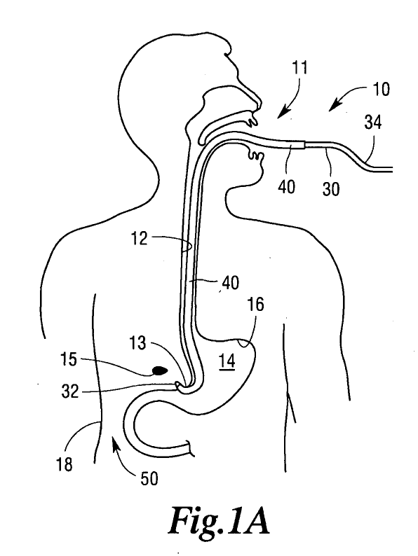

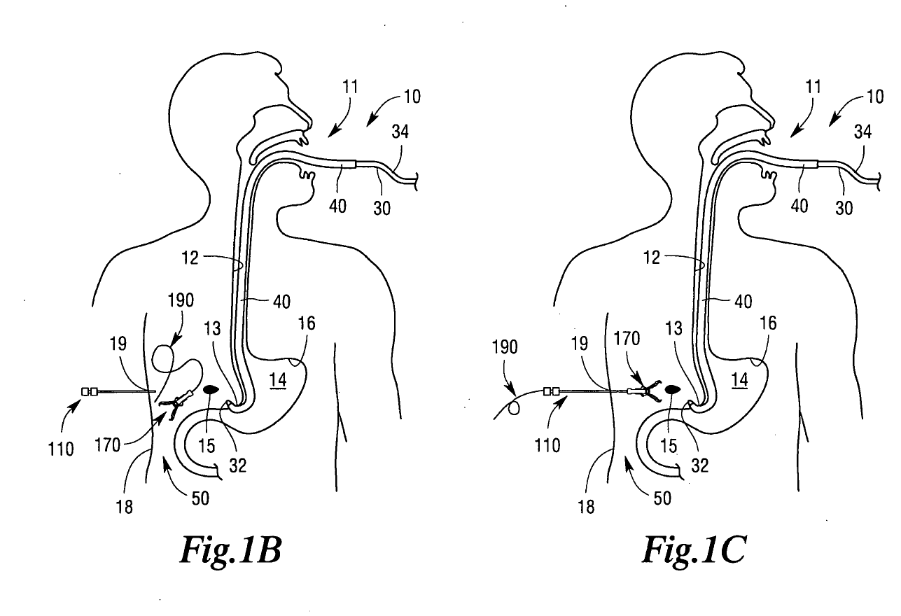

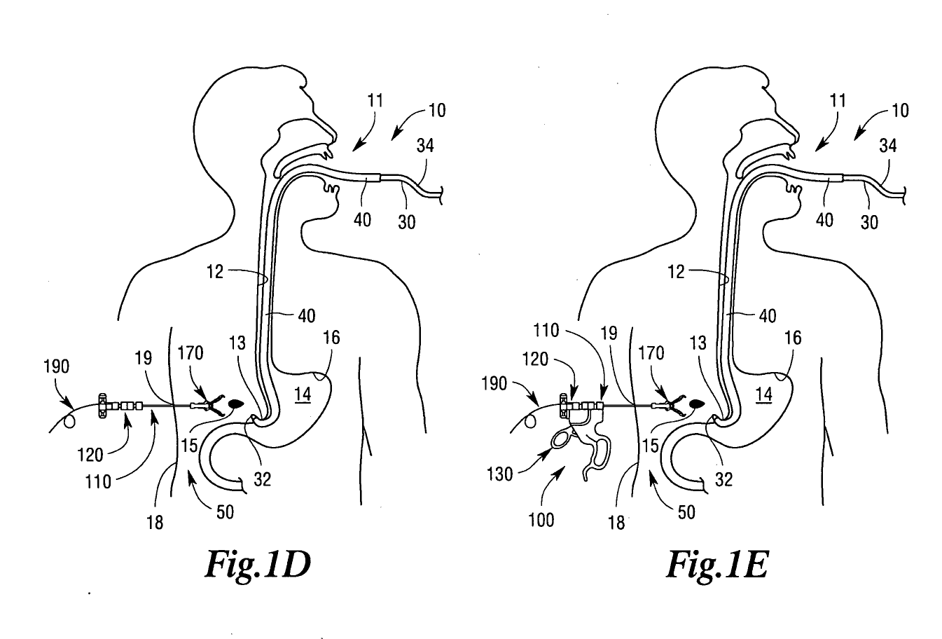

The various embodiments described herein are directed to medical devices and, more particularly, to methods and devices which can be useful in minimally invasive endoscopic procedures carried out with an endoscope and/or a similar surgical instrument. Various embodiments can include methods and devices useful during various medical procedures including, without limitation, methods and devices useful with endoscopes, methods and devices employed through naturally occurring body orifices, and methods and devices related to the assembly of a surgical instrument while at least part of the surgical instrument is inside a patient. Referring now to FIG. 1A, an endoscope 30 is shown inserted into an overtube 40 and inserted through a patient's mouth 11 and esophagus 12 to perform a surgical procedure on a surgical target 15, such as to remove the patient's gall bladder, or perform a cholecystectomy, for example. In various embodiments, overtube 40 and/or endoscope 30 can be inserted through any suitable natural orifice in the patient to form an opening in an organ, or a portion of an organ, such as stomach wall 16, for example. The insertion of the overtube 40 and/or endoscope 30 into the patient may occur trans-orally (as depicted in FIG. 1A), trans-anally, and/or trans-vaginally, for example. In the example depicted in FIG. 1A, the overtube 40 and endoscope 30 are inserted through the mouth 11 and esophagus 12 of the patient and into the stomach 14 to form an opening 13 through the stomach wall 16.

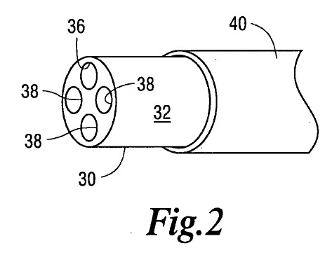

FIG. 2 is a partial perspective view of the distal portion 32 of the flexible endoscope 30 inserted through the overtube 40 of FIG. 1A. A variety of different types of endoscopes are known and, therefore, their specific construction and operation will not be discussed in great detail herein. However, an exemplary, but non-limiting, endoscope and endoscopic system is illustrated and described in U.S. patent application Ser. No. 11/386,861 to Maseda, et al., entitled ENDOSCOPE WORKING CHANNEL WITH MULTIPLE FUNCTIONALITY, the disclosure of which is hereby incorporated by reference in its entirety. In various embodiments, the flexible endoscope 30 has a distal end 32 and a proximal end 34 and may operably support a video camera 36 that communicates with a video display unit that can be viewed by the surgeon during the operation. The flexible endoscope 30 may also comprise one or more working channels 38 extending therethrough for receiving various types of surgical instruments, wherein the working channels 38 may be accessed via working channel ports (not shown) of the endoscope 30.

Focusing now on at least one non-limiting embodiment, a method can be utilized for assembling a surgical instrument inside a patient during a surgical procedure. In various embodiments, as described in greater detail below, an end effector of a surgical instrument may be delivered to a body cavity of the patient, a shaft of the surgical instrument may be inserted through a body wall of the patient and into the body cavity, and the end effector may be releasably connected to the shaft inside the body cavity. In various circumstances, the end effector can be introduced into the body cavity through a natural orifice in the patient and the shaft can be introduced into the body cavity via an opening in the patient created by an incision, for example. This method can provide certain advantages, especially when the end effector has a larger, or wider, diameter than the diameter of the shaft, for example. More particularly, as the wider end effector is not introduced into the body cavity through the same opening as the smaller, or narrower, shaft, the shaft opening can be smaller than would be required if the end effector was inserted through the same opening. Such techniques can result in smaller incisions. Referring now to the exemplary embodiment illustrated in FIGS. 1B-1E, FIG. 1B illustrates an end effector 170 that has been delivered to a body cavity 50 via a first opening, such as a natural orifice of a patient, for example, and a shaft, which may comprise cannula 110, for example, inserted through an incision 19 in a body wall 18 of the patient, wherein, as illustrated in FIG. 1B, the diameter of the end effector 170 is larger than the diameter of the incision 19.

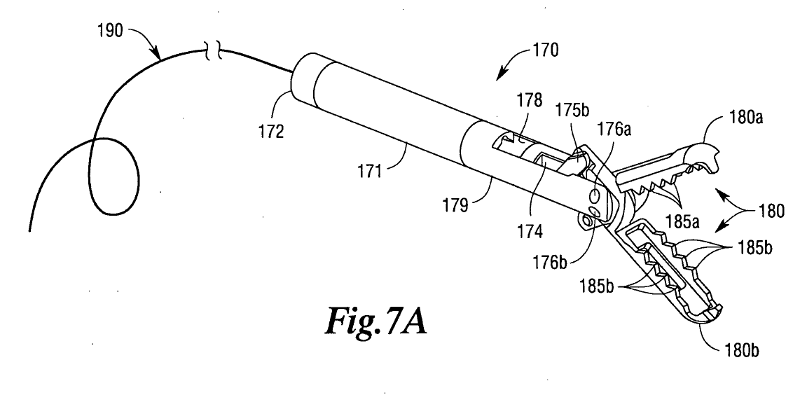

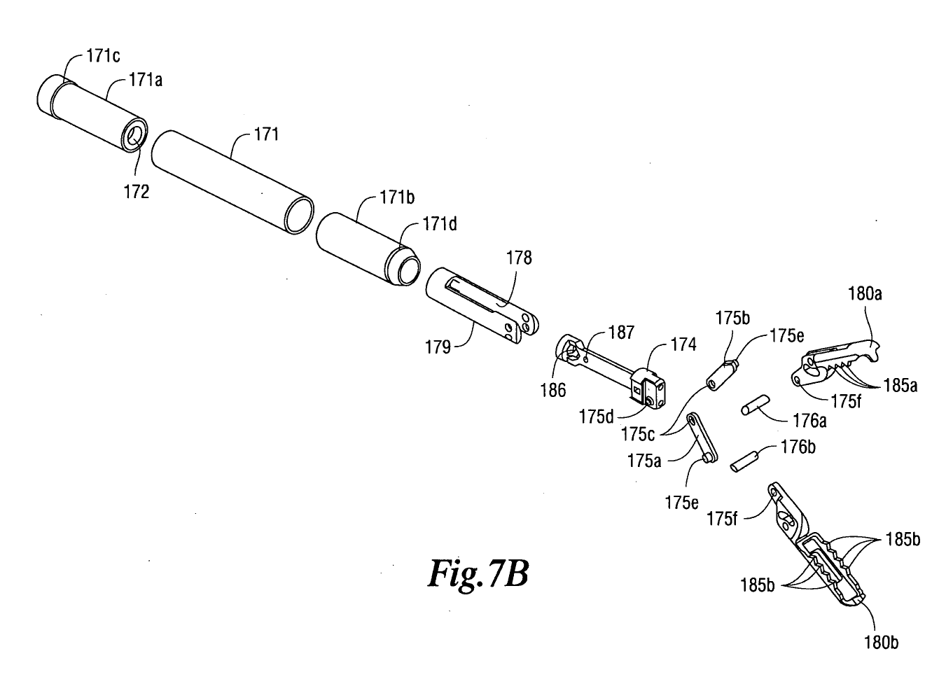

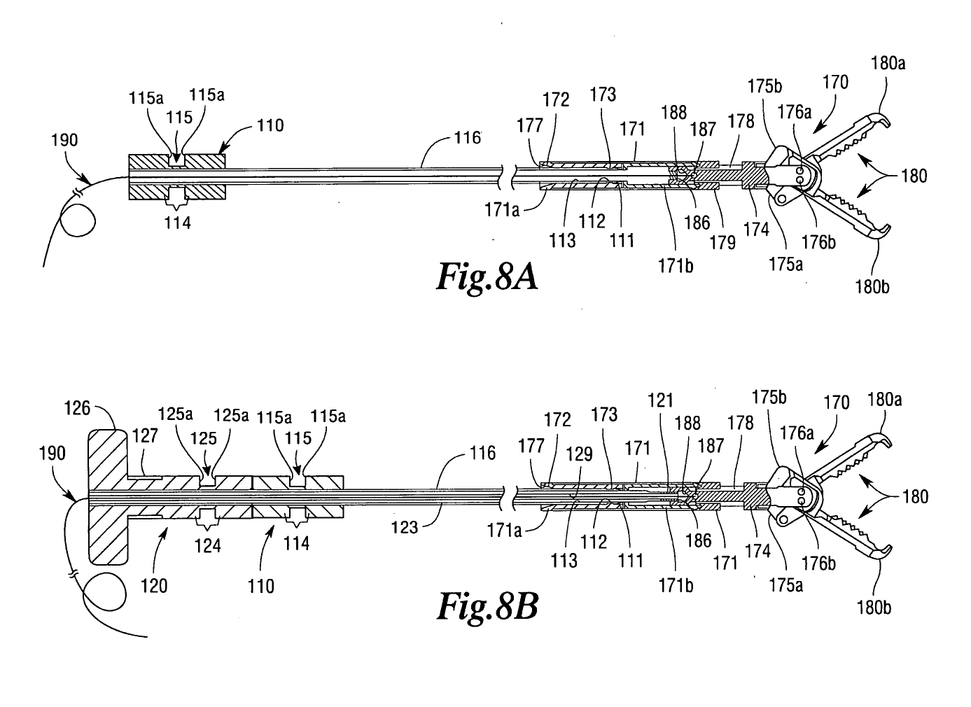

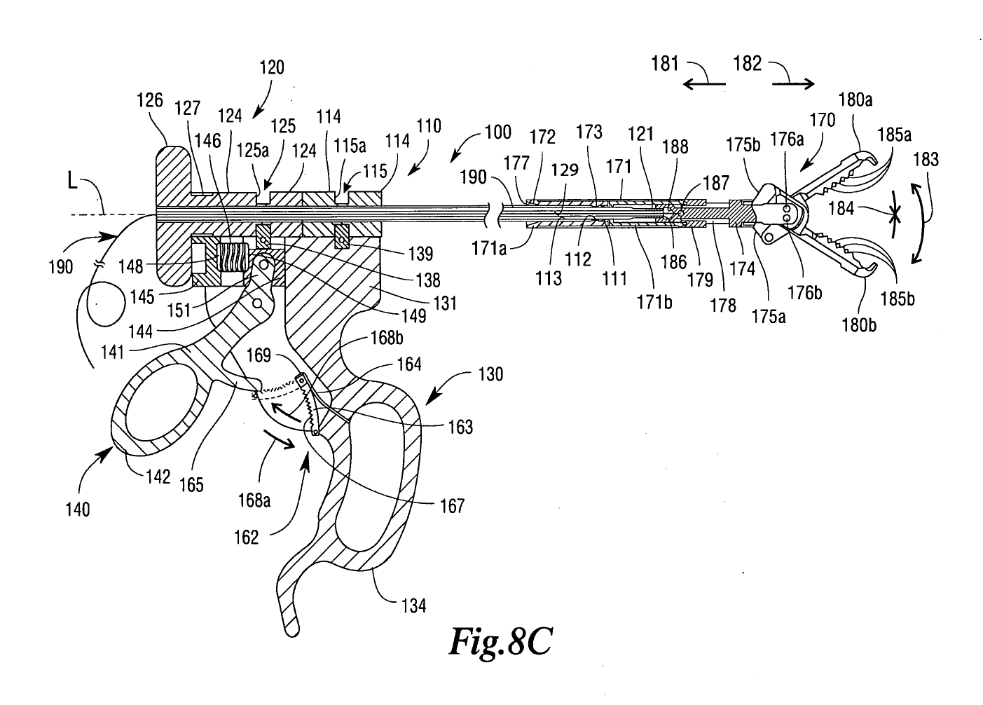

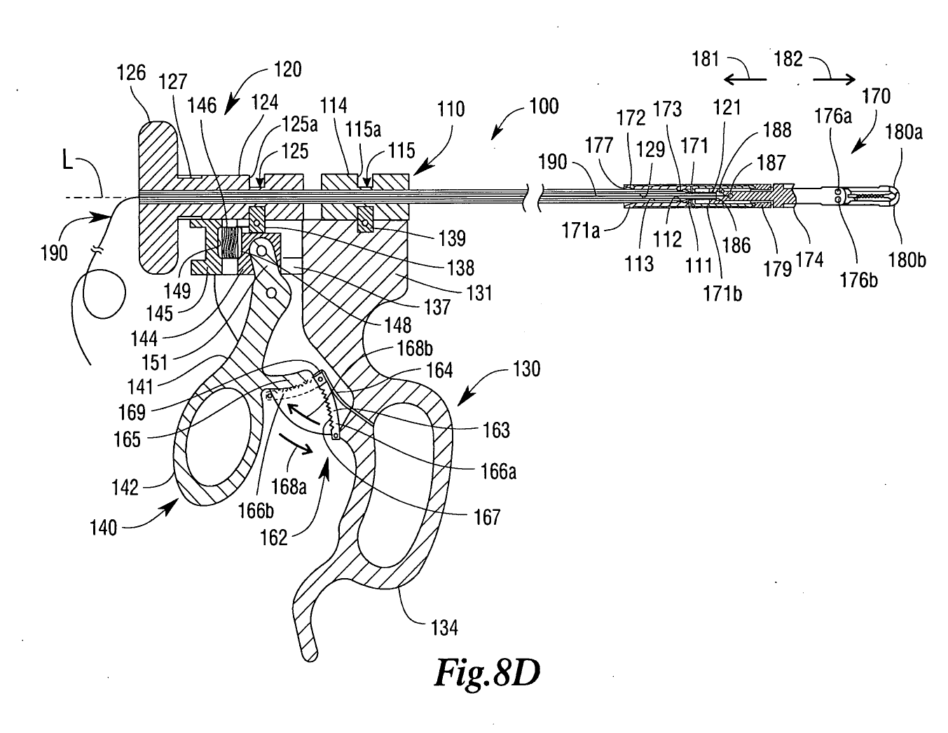

In various embodiments, further to the above, end effector 170 may be releasably connected to the cannula 110 as shown in FIG. 1C. In certain embodiments, end effector 170 may be snap fit, press fit, and/or otherwise suitably engaged with cannula 110. Facilitating such connectability, in at least one embodiment, referring now to FIGS. 7A, 7B, and 8A, end effector 170 may include bushing member 171a and/or bushing member 171b secured within outer housing 171, wherein bushing members 171a and 171b can comprise a receiving orifice 172 extending therethrough which can be configured to receive at least a portion of cannula 110. In certain embodiments, bushing members 171a and/or 171b can be press-fit within housing 171. In at least one embodiment, bushing members 171a and/or 171b can include retention features, such as retention lips 171c and 171d, respectively, which can be configured to engage housing 171 and hold bushing members 171a and 171b in position. In at least one embodiment, bushing member 171a can further comprise one or more radiused and/or beveled surfaces, such as chamfered surface 177 (see FIGS. 8A and 8B), for example, which are sized and configured to assist in positioning and locating cannula 110 in receiving orifice 172 as described in greater detail further below.

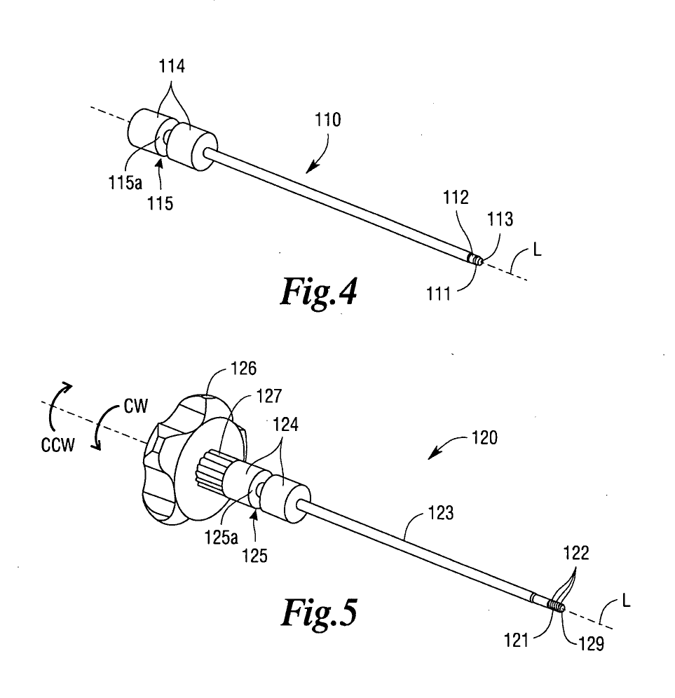

In various embodiments, further to the above, cannula 110 may comprise a body, such as body 116, for example, wherein the body 116 can define a longitudinal axis L and can include a distal end, such as connector portion 111, for example, that is sized and configured to be positioned within the receiving orifice 172 of end effector 170, see FIGS. 4 and 8A. In at least one embodiment, the connector portion 111 can include a recess, such as recess 112, for example, wherein the recess 112 can comprise an annular, or ring-like, indentation, or groove, around at least a portion of the circumference of the cannula 110. Correspondingly, referring to FIGS. 8A and 8B, bushing member 171a may include one or more protrusions, or ridges, such as protrusion 173, for example, that is configured to be received within the cannula recess 112 when the connector portion 111 of cannula 110 is inserted into the receiving orifice 172. In at least one embodiment, bushing member 171a, for example, can be at least partially comprised of a resilient material such that protrusion 173 can be sufficiently compressed to permit the distal end of connector portion 111 to pass thereby and can be sufficiently elastic to allow protrusion 173 to re-expand into recess 112 in cannula 110 once recess 112 is aligned, or at least substantially aligned, with protrusion 173. In certain embodiments, a longitudinal, or at least partially longitudinal, force can be applied to the cannula 110 and/or to the end effector 170 by flexible member 190 in order to press the cannula 110 into the receiving orifice 172 and secure connector portion 111 therein. In certain embodiments, bushing member 171b can comprise a stop which can limit the advancement of cannula 110 within housing 171. In any event, the connector portion 111 of cannula 110 and the bushing member 171a of end effector 170 can form a secure, but releasable connection therebetween.

In various embodiments, as outlined above, an end effector of a surgical instrument can be positioned within a surgical site and a shaft can be inserted into the surgical site such that the end effector can be assembled to the shaft in vivo. In certain embodiments, the end effector can be held in position by a grasper, for example, while the shaft is engaged with the end effector. In various circumstances, however, the end effector may be difficult to grasp and/or hold in position such that a sufficient force can be applied to the shaft and the end effector in order to assemble them together. Described herein are additional embodiments which can allow an end effector, such as end effector 170, for example, and a shaft, such as cannula 110, for example, to be assembled to one another.

In various embodiments, a flexible member, such as flexible member 190, for example, may extend from, may be connected to, and/or may be otherwise operably engaged with the end effector 170. In certain embodiments, the flexible member 190 may be pulled relative to, or through, the cannula 110 such that the end effector 170 can be moved toward cannula 110, and/or such that the cannula 110 and end effector 170 can be moved toward each other, thereby ultimately resulting in the cannula 110 being connected to the end effector 170 as illustrated in FIG. 1C. Flexible member 190 may either be attached to the housing 171 of end effector 170 or, as described in greater detail below, to an actuator of end effector 170 (see FIG. 8A). The flexible member 190 may take the form of a wire, cable, and/or cord for example. In various embodiments, the flexible member 190 may be a stainless steel wire coated in nylon, such as the TyGer™ leader made by TyGer™ Leader Sporting, Ironwood, Mich. Furthermore, in various embodiments, the flexible member 190 may be abrasion resistant, multi-stranded, and/or significantly flexible to enable it to be pulled and/or otherwise manipulated in order to position end effector 170 relative to cannula 110 and to assemble end effector 170 thereto.

In various embodiments, referring to FIG. 4, cannula 110 can further include an aperture, such as aperture 113, for example, which can be defined by inner walls of body 116, for example, and may extend therethrough along longitudinal axis L. Further to the above and referring to FIG. 8A, flexible member 190 and aperture 113 can be configured such that flexible member 190 can be at least partially pulled through aperture 113 and such that end effector 170 can be pulled toward cannula 110. Once receiving orifice 172 of end effector 170 has been aligned with, or at least sufficiently aligned with, connecting portion 111, a longitudinal, or at least substantially longitudinal, force can be applied to end effector 170 in order to press connecting portion 111 into receiving orifice 172 as outlined above. In at least one such embodiment, flexible member 190 can be attached to end effector 170 such that it extends through receiving orifice 172 and, as a result, connecting portion 111 is guided into receiving orifice 172 when end effector 170 is pulled toward connecting portion 111 by flexible member 190. In certain embodiments, cannula 110 can be held stationary while end effector 170 is pulled toward cannula 110 by flexible member 190. In various embodiments, cannula 110 can be pushed toward end effector 170 while, in at least one embodiment, the end effector 170 and the cannula 110 can be moved relatively toward each other resulting in the cannula 110 being connected to the end effector 170. In various embodiments, as outlined above, chamfered surface 177 of receiving orifice 172 may be angled such that connector portion 111 is guided toward receiving orifice 172 and/or such that end effector 170 and cannula 110 become axially aligned, or at least substantially aligned, along longitudinal axis L as shown in FIG. 8A. In any event, the force applied to flexible member 190 can be sufficient to seat connector portion 111 within receiving aperture 172 wherein, in certain embodiments, protrusion 173 can snap into recess 112 thereby forming a releasable connection between cannula 110 and end effector 170.

In various embodiments, referring now to FIGS. 7B and 8C, the end effector 170 may include at least one tissue contacting portion extending from the housing 171. In at least one embodiment, end effector 170 can comprise a tissue contacting portion 180, which may include a first jaw member 180a and a second jaw member 180b. The first and second jaw members 180a,180b may be pivotally coupled to housing 171 by respective first and second pins 176a,176b such that the first and second jaw members 180a, 180b can be rotated between first and second positions. In various embodiments, the first and second positions can comprise fully open and fully closed positions, although embodiments are envisioned in which the first and second jaw members 180a, 180b are moved between partially open and partially closed positions. In any event, in at least one embodiment, the first and second jaw members 180a,180b may be operably engaged with an actuator, such as actuator 174, for example, wherein the actuator 174 can be configured to rotate the first and second jaw members 180a, 180b between their first and second positions. The first jaw member 180a can be coupled to actuator 174 by a first linkage 175a and, similarly, the second jaw member 180b can be coupled to the actuator 174 by a second linkage 175b. More particularly, in at least one embodiment, the first and second linkages 175a, 175b can each comprise a mounting aperture 175c which can be configured to receive mounting pins 175d extending from actuator 174. In addition, the first and second linkages 175a, 175b can each comprise a pivot pin 175e which can be positioned within a pivot aperture 175f in jaw members 180a and 180b, respectively. In use, as described in greater detail below, actuator 174 can be slid along an axis between a proximal position in which the jaw members 180a and 180b are held in a closed configuration and a distal position in which the jaw members 180a and 180b are held in an open configuration, for example.

In various embodiments, referring once again to FIG. 7B, the end effector 170 can further comprise a guide member 179 having a slot 178 configured to define a path for the actuator 174 when it is moved between its proximal and distal positions as described above. More particularly, referring now to FIG. 8A, actuator 174 may be moved reciprocally within slot 178 in the directions indicated by arrows 181 and 182 (FIG. 8C). When the actuator 174 is moved in the direction indicated by arrow 182 (FIG. 8C), the first and second jaw members 180a and 180b can open in the direction indicated by arrow 183. When the actuator 174 is moved in the direction indicated by arrow 181, the first and second jaw members 180a and 180b can close in the direction indicated by arrow 184. In various embodiments, as a result, the first and second jaw members 180a and 180b can cooperate with one another and act like forceps or tongs to grasp and contain tissue, such as dysplastic or cancerous mucosal tissue, for example, therebetween. In certain embodiments, the first and second jaw members 180a and 180b can comprise a plurality of serrations or sets of teeth 185a and 185b, respectively, which can facilitate the grasping of tissue. Although end effector 170 can be utilized in many circumstances, other end effectors can be used. For example, although not illustrated, an end effector can compose a stationary jaw member and a movable jaw member, wherein the movement of an actuator can move the movable jaw member toward and/or away from the stationary jaw member.

In various embodiments, further to the above, a surgical instrument can further comprise an actuation shaft, such as actuation shaft 120, for example (FIG. 1D), and a handle assembly, such as handle 130, for example (FIG. 1E), which may be operably connected to the cannula 110 and/or end effector 170, for example, to form surgical instrument 100 as seen in FIG. 1E. In certain embodiments and referring to FIG. 8B, actuator 174 may further comprise a threaded portion 186 formed in a proximal end of the actuator 174 wherein the threaded portion 186 can be configured to threadably receive a portion of actuation shaft 120. Referring now to FIG. 5, actuation shaft 120 may comprise a body 123 and, in addition, an attachment portion 121 formed on, and/or attached to, a distal end of body 123. In various embodiments, attachment portion 121 and body 123 can be sized and configured such that they can inserted into and extend through aperture 113 of cannula 110, wherein attachment portion 121 can be operably engaged with actuator 174. In at least one such embodiment, attachment portion 121 can comprise threads 122 which can be threadably engaged with the threaded portion 186 of actuator 174 to create a secure and releasable connection between actuation shaft 120 and actuator 174. In various embodiments, inner walls of body 123 may define an aperture, such as lumen 129, for example, which can be sized and configured such that flexible member 190 may pass therethrough as described in greater detail further below.

In order to assemble surgical instrument 100, as discussed above, the end effector 170 can be positioned in a body cavity through a first opening, such as a natural orifice, in the patient and the cannula 110 can be inserted into the body cavity through a second opening in the patient. As also discussed above, the end effector 170 can comprise a flexible member 190 mounted thereto wherein the flexible member 190 can be pulled through aperture 113 in cannula 110 in order to align and mount end effector 170 to cannula 110. In various embodiments, referring to FIG. 8A, flexible member 190 may be tethered to actuator 174 via a hole 187. In certain embodiments, at least a portion of flexible member 190 can be secured within the hole 187 by a fastener, for example. In at least one embodiment, flexible member 190 may be glued, welded, tied, and/or otherwise attached to actuator 174. In any event, in order to pull flexible member 190 into aperture 113, a grasper, for example, can be inserted through aperture 113 from outside of the patient and into the surgical site such that the flexible member 190 can be grasped and pulled into aperture 113. In various embodiments, flexible member 190 can have a sufficient length such that the flexible member 190 can extend entirely through aperture 113 and such that an end of flexible member 190 can be positioned outside of the cannula 110. In other embodiments, the flexible member 190 may only have a length sufficient to extend partially into cannula 110, for example.

In various embodiments, referring to FIG. 8A, flexible member 190 can be pulled through aperture 113 of cannula 110 before actuation shaft 120 is inserted into cannula 110. In at least one such embodiment, referring now to FIGS. 5 and 8B, at least a portion of the flexible member 190 can be inserted into lumen 129 of actuation shaft 120 such that actuation shaft 120 can be slid down, or along, flexible member 190 until attachment portion 121 of actuation shaft 120 is engaged with threaded portion 186 of actuator 174 as outlined above. In other embodiments, at least a portion of actuation shaft 120 may be inserted into aperture 113 of cannula 110 before flexible member 190 is pulled through aperture 113. In at least one such embodiment, a grasper, for example, can be inserted through lumen 129 of actuation shaft 120, wherein the grasper can be used to grasp flexible member 190 and pull it through lumen 129. In certain embodiments, attachment portion 121 of actuation shaft 120 may be secured to actuator 174 before flexible member 190 is pulled therethrough. In at least one such embodiment, the attachment portion 121 may be releasably attached to actuator 174 by positioning the distal end of attachment portion 121 in threaded aperture 186 of actuator 174 and rotating actuation shaft 120 in a clockwise direction (in the direction of arrow CW in FIG. 5), for example, such that the threads 122 of attachment portion 121 engage the threads of aperture 186, see FIG. 8B. In at least one embodiment, referring to FIG. 5, actuation shaft 120 may further comprise an enlarged portion, or knob 126, for example, which can be configured to facilitate the rotation of actuation shaft 120.

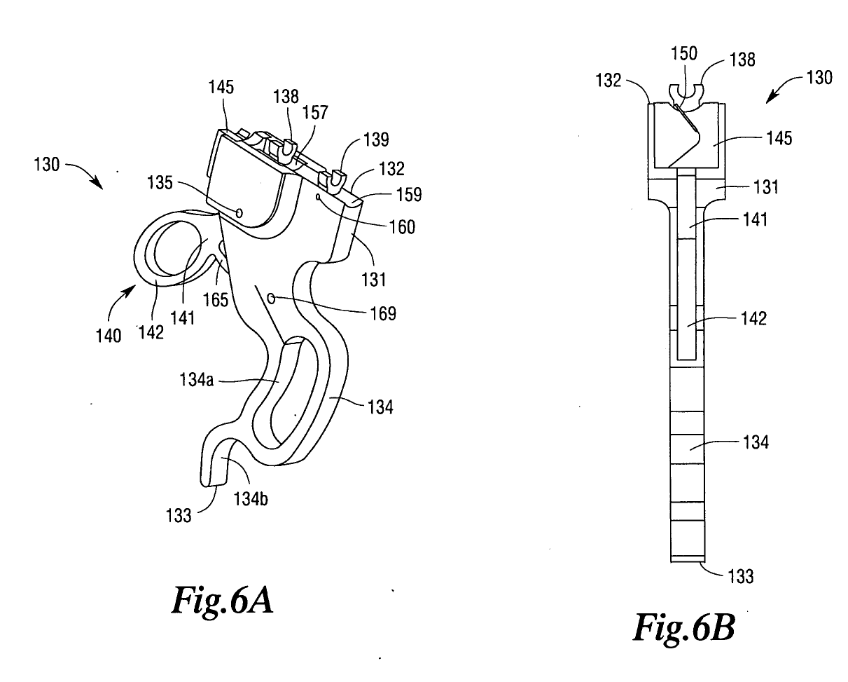

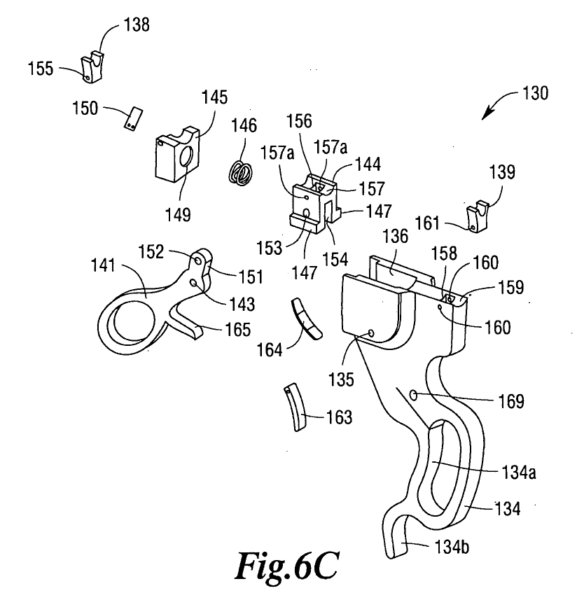

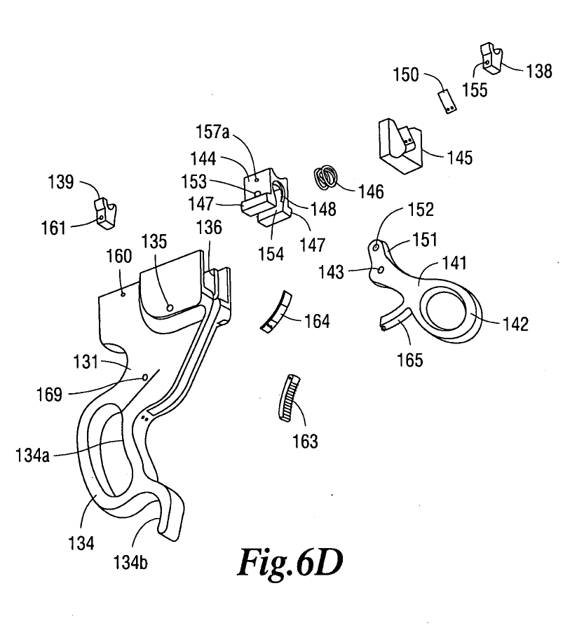

In various embodiments, as indicated above, surgical instrument 100 can further comprise a handle assembly, such as handle 130 (FIG. 6A), for example, which can be configured to motivate actuation shaft 120 and actuator 174 and, accordingly, move jaw members 180a and 180b between their first and second positions, for example. Referring now to FIGS. 6A-6D, handle 130 may comprise a housing 131 and, in addition, a trigger assembly, such as trigger assembly 140, for example, movably coupled to and extending from the housing 131. In various embodiments, as described in greater detail further below, the trigger assembly 140 can further comprise one or more attachment members which can attach and operably engage the actuation shaft 120 to the trigger assembly 140. In any event, once actuation shaft 120 has been operably engaged with trigger assembly 140, in at least one embodiment, trigger assembly 140 can be actuated, or moved toward a grip 134, in order to pull actuation shaft 120, and actuator 174 attached thereto, in a proximal direction, for example. When actuator 120 is pulled in a proximal direction, jaw members 180a and 180b can be pivoted inwardly into a closed position. In at least one such embodiment, the trigger assembly 140 can be released, or pushed away from grip 134, such that actuation shaft 120 and actuator 174 are pushed distally. When actuator 174 is moved distally, jaw members 180a and 180b can be pivoted outwardly into an open position. In various other embodiments, although not illustrated, an actuator can be moved distally in order to close an end effector and can be moved proximally in order to open the end effector. In any event, various details of handle 130 and surgical instrument 100 are discussed below.

Referring primarily to FIG. 6A, the handle 130 may provide an ergonomic interface for a user to operate the handle 130 and/or surgical instrument 100 once assembled. Housing 131 may comprise a top portion 132 and a bottom portion 133. Formed in the bottom portion 133 may be a finger grip, such as finger grip 134, for example. Finger grip 134 may include an upper finger rest 134a configured to support at least one finger of a user gripping the handle 130 and a lower finger rest 134b which can also configured to support at least one finger of the user. The top portion 132 may comprise one or more connection members, such as snap yoke 139, for example, extending therefrom which can be configured to support and retain cannula 110 in position. In various embodiments, referring to FIGS. 8A-8D, cannula 110 can comprise a connecting portion 115 which can be configured to be received, press-fit, and/or snap-fit within the snap yoke 139. More particularly, in at least one embodiment, connecting portion 115 can comprise a retention groove, or slot, 115a which can be configured to be positioned within the snap yoke 139. In addition, the connecting portion 115 can comprise one or more retention shoulders, or support members, 114 which can be configured to co-operate with snap yoke 139 to releasably retain cannula 110 in position.

In at least one embodiment, snap yoke 139 can comprise one or more resilient, or elastic, arms which can be configured to flex outwardly as connecting portion 115 is inserted therein and resiliently move, or snap, inwardly into retention slot 115a as connecting portion 115 is seated within snap yoke 139. In at least one such embodiment, snap yoke 139 can be comprised of plastic, for example. In certain embodiments, snap yoke 139 can be configured to at least partially permanently deform when connecting portion 115 is inserted therein. In various embodiments, snap yoke 139, retention slot 115a, and retention shoulders 114 can be sized and configured to prevent, or at least limit, relative movement between cannula 110 and handle 130. In certain embodiments, these features can be configured such that there is little, if any, relative longitudinal movement between handle 130 and cannula 110 along the longitudinal axis L of cannula 110.

In various embodiments, further to the above, snap yoke 139 may be secured to handle housing 131 by a pin, and/or any other suitable fastener. In certain embodiments, snap yoke 139 can be welded to, integrally formed with, and/or otherwise suitably secured to housing 131. Although only one snap yoke 139 is illustrated in the exemplary embodiment, a plurality of snap yokes can be utilized. Furthermore, although one or more snap yokes may be used, other connection members, such as any suitable clips, clamps, ties, and/or straps, for example, can be utilized to mount cannula 110 to handle 130 in lieu of the snap yokes or, alternatively, in combination with the snap yokes. In any event, the snap yokes, and/or the other suitable connection members, can allow the cannula 110 to be easily assembled to, and easily disassembled from, handle 130.

In various embodiments, housing 131 of handle 130 may also comprise first pin holes 135 formed therein for pivotably supporting a part of trigger assembly 140. Referring primarily to FIGS. 6A and 6C, trigger assembly 140 may comprise a body 141 having a thumb grip, such as thumb grip 142, for example, formed therein. Trigger body 141 may be pivotably engaged with handle housing 131 at handle pin holes 135 via trigger pin hole 143 formed in trigger body 141. A pivot pin (not shown) may pass through handle pin holes 135 and through trigger pin hole 143, wherein, in at least one embodiment, the pivot pin may be may be clipped, press-fit, and/or otherwise secured within pin holes 135. In various embodiments, as a result, trigger body 141 may rotate with respect to handle housing 131 about an axis defined by handle pin holes 135.

Referring primarily to FIGS. 6C and 6D, trigger assembly 140 may further comprise a movable sled 144 and a stop 145, wherein the movable sled 144 may be sized and configured to translate within a channel 136 formed in handle housing 131. In various embodiments, channel 136 can be sized and configured to guide sled 144 along a predetermined path, wherein, although not illustrated, channel 136 may include one or more slots defined therein which can be configured to receive rails 147 extending from sled 144 such that sled 144 can be translated in longitudinal directions within handle housing 131. Although trigger assembly 140 can be configured to move sled 144 between predetermined first and second positions, one or more stops, such as stop 145, for example, can be positioned within or relative to channel 136 such that stop 145 can limit the travel of sled 144. In various embodiments, stop 145 may be glued, welded, or otherwise attached to housing 131 at an end of the channel 136. In at least one embodiment, a variable load generating member, such as spring 146, for example, may be positioned intermediate movable sled 144 and stop 145. In certain embodiments, the variable load generating member can comprise a wave spring. In at least one embodiment, spring 146 can comprise a tension spring, a coil spring, a compression spring, a torsion spring, and/or an elastic core, for example. In various embodiments, sled 144 and/or stop 145 can comprise one or more alignment, and/or mounting, features which can align and/or retain spring 146 in position. In at least one such embodiment, referring again to FIGS. 8C and 8D, sled 144 may comprise a first lip 148 and stop 145 may comprise a second lip 149, wherein the first and second lips 148 and 149 can be configured to engage spring 146. In at least one embodiment, spring 146 can be configured to bias the movable sled 144 in a distal direction, i.e., in the direction of arrow 182 (FIG. 8C), for example, such that the jaw members of the end effector 170 are biased into an open configuration. Although not illustrated, other embodiments are envisioned in which a spring is positioned intermediate sled 144 and a portion of housing 131 to bias sled 144 in a proximal direction such that the jaw members are biased into a closed configuration.

In various embodiments, further to the above, the trigger body 141 may be operably engaged with the sled 144 such that the movement of trigger body 141 is transmitted to sled 144. In certain embodiments, referring primarily to FIGS. 6C and 6D, the trigger body 141 may further include a lever arm 151 extending therefrom and, in addition, a lever pin hole 152 in lever arm 151. Movable sled 144 may further comprise sled apertures 153 extending therethrough and, in addition, an inner slot 154 that is sized and configured to receive a portion of lever arm 151. In at least one such embodiment, apertures 153 can be aligned with pin hole 152 when lever arm 151 is positioned within inner slot 154. A second pivot pin (not shown) may pass through sled apertures 153 and lever pin hole 152 such that the movement of trigger body 141 can be transmitted to sled 144. In certain embodiments, apertures 153 can comprise elongated and/or enlarged slots which can provide one or more camming surfaces against which the second pivot pin mounted to trigger body 141 can act, or bear, against. More particularly, in at least one such embodiment, the second pivot pin may traverse an arcuate path when it is moved by trigger body 141, wherein the elongated slots or camming surfaces can be permit relative sliding movement between the second pivot pin and the sled 144 while still permitting sled 144 to be moved proximally and/or distally within trigger assembly 140.

In various embodiments, further to the above, trigger body 141 can be moved toward grip 134 in order to move sled 144 proximally and, as a result, pull actuation shaft 120 proximally as well. As outlined above, referring again to FIG. 6A, the sled 144 can comprise one or more connection members which can be configured to mount actuation shaft 120 to sled 144. In various embodiments, a connection member can comprise a snap yoke, such as snap yoke 138, for example, which may be secured to, attached to, and/or integrally formed with movable sled 144. Referring to FIGS. 8C and 8D, snap yoke 138 can be sized and configured to releasably receive at least a portion of actuation shaft 120, such as connecting portion 125, for example, therein. In various embodiments, snap yoke 138 can comprise one or more resilient, or elastic, arms which can be configured to flex outwardly as connecting portion 125 is inserted therein and resiliently move, or snap, inwardly into a retention slot 125a as connecting portion 125 is seated within snap yoke 138. In at least one such embodiment, connecting portion 125 can be configured to be press-fit, or snap-fit, within snap yoke 138 such that there is little, if any, relative movement between connecting portion 125 of actuation shaft 120 and snap yoke 138, especially along the longitudinal axis L of actuation shaft 120. Although only one snap yoke 138 is illustrated in the exemplary embodiment, a plurality of snap yokes can be engaged with sled 144 which can be configured to drive actuation shaft 120 between its first and second positions. In various embodiments, actuation shaft 120 can comprise a plurality of retention slots, which can be configured to be positioned within the snap yokes, for example, and can include one or more drive shoulders, such as support members 124, for example, which can be configured to provide a bearing surface between the snap yokes and actuation shaft 120. The support members 124 can also prevent, or at least inhibit, relative longitudinal movement between actuation shaft 120 and sled 144. Although one or more snap yokes may be used, other connection members, such as any suitable clips, clamps, ties, and/or straps, for example, can be utilized to mount actuation shaft 120 to sled 144 in lieu of the snap yokes or, alternatively, in combination with the snap yokes. In any event, the snap yokes, and/or the other suitable connection members, can allow the actuation shaft 120 to be easily assembled to, and easily disassembled from, sled 144.

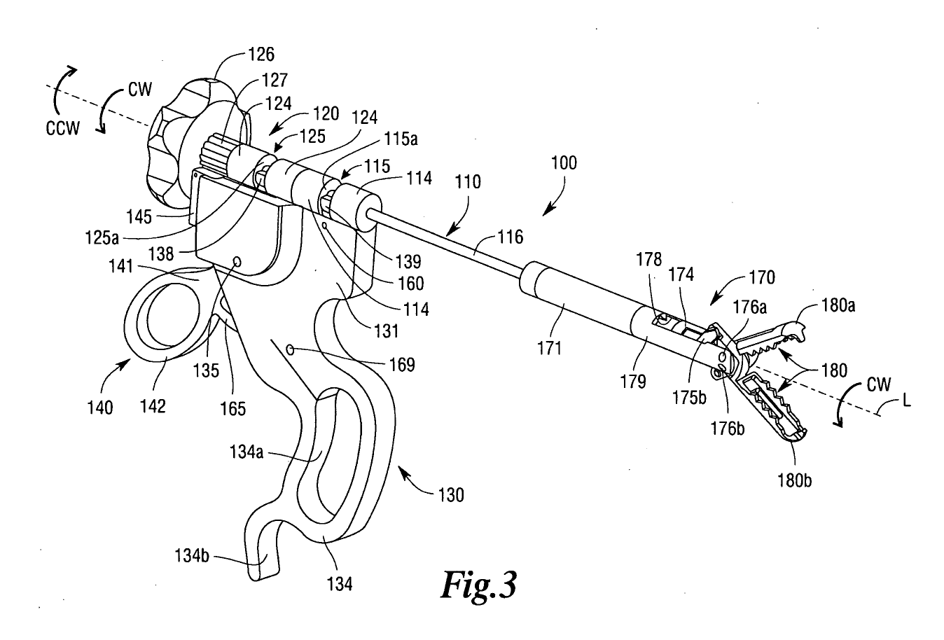

In various embodiments, referring still to FIG. 6A and also to FIGS. 3 and 8C, further to the above, first support surface 157 of movable sled 144 can at least partially support and/or cradle support members 124 of actuation shaft 120 when actuation shaft 120 is attached to snap yoke 138. Similarly, second support surface 159 of handle housing 131 can at least partially support and/or cradle support members 114 of cannula 110 when cannula 110 is attached to snap yoke 139. As outlined above, connecting portions 125, 115 may be press fit into snap yokes 138, 139, respectively, such that cannula 110 can be held in position and actuation shaft 120 can be moved relative to cannula 110. In various embodiments, cannula 110 can be mounted to trigger housing portion 131 and actuation shaft 120 can be mounted to sled 144 sequentially. In other various embodiments, cannula 110 and actuation shaft 120 can be mounted to trigger housing portion 131 and sled 144 at the same time, or at least at substantially the same time. More particularly, in at least one embodiment, snap yokes 138, 139 may be positioned with respect to each other such that, after actuation shaft 120 is inserted through cannula 110, as described above, both actuation shaft 120 and cannula 110 may be connected to handle 130 at approximately the same time. In at least one such embodiment, snap yokes 138 and 139 can be configured to hold and align actuation shaft 120 and cannula 110 such that they are concentrically, or at least substantially concentrically, aligned with one another. Stated another way, the snap yokes 138 and 139 can be configured such that the longitudinal axis of actuation shaft 120 is collinear, or at least nearly collinear, with the longitudinal axis of cannula 110.

In various embodiments, referring primarily to FIG. 6C, snap yoke 138 may be attached to sled 144 in a sled recess 156 formed in the first support surface 157 of the movable sled 144. In at least one embodiment, snap yoke 138 may include a first hole 155 and, in addition, movable sled 144 may include a pair of holes 157a extending through sled 144 and into sled recess 156, wherein first hole 155 and holes 157a can be configured to receive a first set pin (not shown) therein in order to secure snap yoke 138 to sled 144. Similarly, snap yoke 139 may likewise be attached to handle housing 131 in a housing recess 158 formed in the second support surface 159 of the housing 131. A second set pin (not shown) may pass through a pair of holes 160 in housing 131 and through a second hole 161 in second snap yoke 139 in order to secure snap yoke 139 to housing 131. While set pins can be utilized to secure snap yokes 138, 139 to the trigger assembly 140 and housing 131, respectively, any suitable fastener or form of attachment may be utilized, such as gluing and/or welding, for example.

In use, referring to FIGS. 8C-8D, a user may grip handle 130 at finger grip 134 and thumb grip 142 of the trigger assembly 140. For example, a user's thumb may be supported in thumb grip 142 such that the user may also place at least one finger in finger grip 134 on handle housing 131. The user, gripping the handle 130 in such a fashion, may articulate the trigger assembly 140 by squeezing his or her hand so that trigger body 141 moves with respect to handle housing 131. When the trigger body 141 rotates about handle pin holes 135 and toward finger grip 134, movable sled 144 is driven proximally, i.e., in the direction of arrow 181, thereby compressing spring 146 (see FIG. 8D). When the user relaxes his or hand, the spring 146 can bias the movable sled 144 distally, i.e., in the direction of arrow 182 (see FIG. 8C). By this point, the reader should appreciate that when trigger assembly 140 is actuated, snap yoke 138 moves with respect to snap yoke 139 and, accordingly, actuation shaft 120 is moved with respect to cannula 110. While the foregoing describes at least one embodiment where the trigger assembly 140 is operated by a user's thumb, the trigger assembly and housing may alternatively be designed such that the trigger assembly is operated by any other suitable finger or fingers, for example.

In various embodiments, referring now to FIGS. 1B-1E and 8C, a kit comprising end effector 170, flexible member 190, cannula 110, actuation shaft 120, and handle 130 may be assembled by a user to form surgical instrument 100 as follows. First, the flexible member 190 can be passed through the cannula 110 via aperture 113. Second, the flexible member can be pulled until the end effector 170 connects to the cannula 110 via connector portion 111 (see FIG. 1C). Third, the flexible member 190 can be inserted through actuation shaft 120 via lumen 129. Fourth, the actuation shaft 120 can be translated along the flexible member 190, through aperture 113 of cannula 110, and into end effector 170. Fifth, the attachment portion 121 of actuation shaft 120 can be coupled to actuator 174 of end effector 170 by rotating knob 126 such that threaded attachment portion 121 engages threaded portion 186 of actuator 174 (see. FIG. 1D). Sixth, the first connecting portion 125 of actuation shaft 120 and the second connecting portion 115 of cannula 110 can be releasably attached to handle 130 at first and second snap yokes 138, 139, respectively, thereby forming surgical instrument 100 (see. FIG. 1E). While the above provided order of steps can be utilized to assemble a surgical instrument, various other steps can be inserted between the enumerated steps and/or the order of the steps can be rearranged as appropriate. For example, as outlined above, the step of inserting actuation shaft 120 into cannula 110 can occur before the step of passing the flexible member 190 into the cannula 110.

As outlined above, cannula 110 can be inserted through a body cavity of the patient through an incision before the flexible member 190 is pulled through cannula 110. In various other embodiments, however, the flexible member 190 can be pulled through an incision in the patient before the cannula is inserted into the incision. In at least one such embodiment, the end effector 170 and flexible member 190 can be inserted into a body cavity and an incision can be made in the patient such that a grasper, for example, can be inserted through the incision in order to grasp the flexible member 190. Thereafter, the grasper can be pulled proximally such that at least a portion of the flexible member 190 is positioned outside of the patient's body, for example, wherein at least a portion of the flexible member 190 can then be fed up through the aperture 113 in cannula 110. In at least one such embodiment, the grasper can be sized and configured such that it can be passed through aperture 113 and, as a result, pull flexible member 190 into aperture 113 as well. In any event, the cannula 110 can be moved toward the patient along the flexible member 190 until at least a portion of the cannula 110 enters into the body cavity such that the end effector 170 can be attached to the cannula 110 as outlined above. The subsequent steps of assembling the various components of surgical instrument 100 can parallel, or at least substantially parallel, those steps described above.

Referring now to FIGS. 6C-6D and 8C-8D, the jaw members of end effector 174 may be configured such that they can be locked into a closed, actuated position (FIG. 8D) by a trigger lock 162 operably engaged with handle housing 131. In various embodiments, trigger lock 162 may comprise a pivotable lever 163 that includes a set of teeth 167 sized and configured to engage a notched arm 165 extending from trigger body 141. Pivotable lever 163 may be mounted to handle housing 131 via pivot pin holes 169 and a pivot pin (not shown) extending through pivot pin holes 169. Trigger lock 162 may further comprise a biasing member, such as leaf spring 164, for example, mounted to handle housing 131, wherein leaf spring 164 can be biased against a portion of pivotable lever 163 such that lever 163 can be biased from an unlocked position (shown in solid lines) into a locked position (shown in phantom lines). When the teeth 167 of lever 163 are engaged with arm 165, the teeth 167 can prevent trigger body 141 from being moved into its closed position.

In various embodiments, referring now to FIG. 3, various portions of assembled surgical instrument 100 can be rotated about an axis, such as longitudinal axis L, for example, in order to adjust the orientation of end effector 170. More particularly, in at least one embodiment, a sub-assembly comprising cannula 110, end effector 170, flexible member 190, and actuation shaft 120 can be configured such that it can be rotated relative to handle 130. As the reader will recall, cannula 110 is mounted to handle housing 131 by a snap yoke 139 and, in addition, actuation shaft 120 is mounted to sled 144 by a snap yoke 138. In at least one such embodiment, the cannula 110 can be rotated within snap yoke 139 and, similarly, the actuation shaft 120 can be rotated within snap yoke 138. Owing to the concentric, or at least substantially concentric, alignment of cannula 110 and actuation shaft 120, cannula 110 and actuation shaft 120 can be rotated within their respective snap yokes at the same time. Furthermore, the connecting portion 115 of cannula 110 and the connecting portion 125 of actuation shaft 120 can comprise concentric, or at least substantially concentric, cylindrical members which can be configured to rotate within support surfaces 159 and 157, respectively. In at least one such embodiment, support surfaces 159 and 157 can be contoured to permit relative sliding movement between the connecting portions 115 and 125 and support surfaces 159 and 157, respectively.

In order to facilitate the rotation of actuation shaft 120 and actuator 110, referring to FIG. 3, surgical instrument 100 can further comprise a knob, such as knob 126, for example, which can be mounted to, assembled to, and/or integrally-formed with actuation shaft 120 (see also FIG. 5). Rotating the knob 126 in a direction indicated by arrow CW can cause the body 123 and attachment portion 121 of actuation shaft 120 to also rotate in the direction indicated by arrow CW, see FIGS. 3, 5, and 8C. Further to the above, rotating threaded attachment portion 121 in direction CW can advance attachment portion 121 into threaded portion 186 of actuator 174 until the attachment portion 121 contacts or bottoms out against an inner surface 188 of actuator 174. Further rotation of knob 126 in the direction of arrow CW can tighten the connection between attachment portion 121 of actuation shaft 120 and threaded portion 186 of end effector 170, especially when cannula 110 is held in position. More particularly, as the reader will recall, the end effector 170 is engaged by both the cannula 110 and the actuation drive shaft 120, wherein, as a result, the actuation shaft 120 can be securely tightened to the threaded portion of actuator 174 when the end effector 170 is held stationary by the cannula 110. In various circumstances, the cannula 110 can be held in position by the surgeon by placing a thumb, for example, on connecting portion 115, for example. In any event, once actuation shaft 120 has been sufficiently tightened to actuator 174, the surgeon can release cannula 110 and the rotation of knob 126 in the direction indicated by arrow CW can cause end effector 170 to rotate about longitudinal axis L in the direction indicated by arrow CW. In certain embodiments, although not illustrated, the surgical instrument 100 can further comprise a lock which holds cannula 110 in place while actuation shaft 120 is rotated relative to cannula 110.

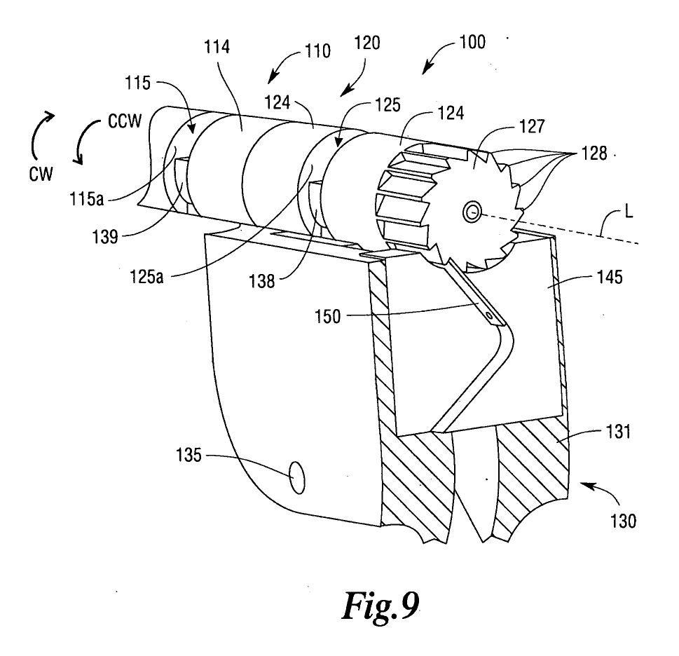

In various embodiments, referring still to FIG. 3, rotation of end effector 170 in the direction of arrow CCW may result in actuation shaft 120 being loosened with respect to or disengaged from end effector 170. While, as explained below, this may be desirable when disassembling the instrument for removal from a patient, it may be undesirable while a user is operating with the assembled surgical instrument 100. Therefore, in various embodiments, it may be desirable to prevent the user from rotating knob 126 in the direction of arrow CCW while the instrument 100 is fully assembled and is being used. In various embodiments, a ratcheting mechanism may be provided which can be configured to permit actuation shaft 120 to rotate in a direction indicated by arrow CW but prevent actuation shaft 120 from rotating in a direction indicated by arrow CCW. In at least one such embodiment, referring now to FIGS. 5 and 9, actuation shaft 120 may further include a ratchet wheel 127 mounted thereto, and/or integrally-formed therewith, wherein, in at least one embodiment, ratchet wheel 127 can be positioned between knob 126 and handle housing 131. In such embodiments, a pawl member can be mounted to handle housing 131 wherein the pawl can be configured to slide over the teeth of ratchet wheel 127 when the ratchet wheel 127 is rotated in direction CW but bite into, or engage, the teeth of the ratchet wheel 127 when the ratchet wheel 127 is rotated in direction CCW. In at least one embodiment, the pawl member can comprise a spring, such as leaf spring 150, for example, having one end mounted to stop 145, for example, and a second end engaged with ratchet wheel 127 wherein leaf spring 150 may operably engage the teeth 128 of ratchet wheel 127 and function as a pawl of a ratchet. Although such embodiments are entirely suitable for their intended purpose, other ratcheting mechanisms or designs are possible to allow the rotation of actuation shaft 120 in one direction while preventing or hindering the rotation of actuation shaft 120 in another direction.

Referring now to FIGS. 3 and 8A-C, in various embodiments, the removal of surgical instrument 100 from a surgical site may occur as follows. First, actuation shaft 120 can be decoupled from end effector 170 by rotating knob 126 in the direction of arrow CCW (see FIG. 3) such that attachment portion 121 disengages from threaded portion 186 of actuator 174. In such circumstances, the interface between cannula 110 and end effector 170 can provide sufficient friction to hold end effector 170 in position while actuation shaft 120 is being disengaged from end effector 170. In various circumstances, the actuation shaft 120 can then be pulled proximally and removed from aperture 113 of cannula 110 or, alternatively, the actuation shaft 120 can remain positioned within the cannula 110 while subsequent disassembly steps are performed. In any event, the end effector 170 can then be disconnected from the cannula 110. In various embodiments, a plunger can be inserted through aperture 113 of cannula 110 in order to engage end effector 170 and slide it off of the end of cannula 110 while, in certain embodiments, the actuation shaft 120, once unthreaded from end effector 170, can be utilized to push end effector 170 off of cannula 110. In either event, the end effector 170 can be pushed distally, i.e., in the direction of arrow 182 (see FIG. 8B), while the cannula 110 can be securely held such that the attachment portion 121 of actuation shaft 120 can be pressed against the threaded portion 186 of actuator 174, for example, to disconnect end effector 170 from connector portion 111 of cannula 110. Alternatively, the cannula 110 can be used to pull end effector 170 against a body wall 18 (see FIG. 1C) such that end effector 170 can decouple from cannula 110. After end effector 170 has been disengaged from cannula 110, cannula 110 can be withdrawn from the surgical site by removing it from the opening through which it was inserted and, in addition, the end effector 170 can be withdrawn from the surgical site by removing it through the opening through which it was inserted. As outlined above, the cannula 110 can be removed from the surgical site through a first opening, such as a natural orifice or an incision, for example, and the end effector 170 can be removed from the surgical site through a larger second opening, such as a natural orifice or incision, for example. In the circumstances where cannula 110 is removed from the surgical site before the end effector 170 is removed, cannula 110 can be slid proximally along flexible member 190. In the circumstances where the end effector 170 is removed from the surgical site before the cannula 110 is removed, the end effector 170 and/or at least a portion of the flexible member 190 can be grasped in order withdraw the flexible member 190 from the cannula 110. In any event, when removing the end effector 170 from a patient, the end effector 170 may be removed from the patient through overtube 40 by using a grasper (not shown) inserted through a working channel 38 of endoscope 30 (see FIGS. 1B and 2) to grab flexible member 190 and pull flexible member 190 and, accordingly, end effector 170 out of body cavity 50 through overtube 40. In various circumstances, owing to the coupling between flexible member 190 and end effector actuator 174, the end effector 170 may be at least partially closed (see, e.g., FIG. 8D) when flexible member 190 is pulled which can facilitate its passage through the overtube 40. In various embodiments, further to the above, handle 130 can be decoupled from the actuation shaft 120 and cannula 110 before the actuation shaft 120 and the cannula 110 are disengaged from the end effector 170 as discussed above. Referring to FIG. 8B, actuation shaft 120 and cannula 110 can be detached from handle 130 by pulling shaft 120 and cannula 110 away from handle 130 until snap yokes 138, 139 are disengaged from shaft 120 and cannula 110, respectively. In other embodiments, the handle 130 can remain engaged with the actuation shaft 120 and/or the cannula 110 while the actuation shaft 120 and/or the cannula 110 are disengaged from the end effector 170. While the sequence of steps provided above can be utilized, various other steps can be inserted between the enumerated steps and/or the order of the steps can be rearranged as appropriate to permit the in vivo disassembly of an end effector from a surgical instrument.

In various embodiments, in view of the above, a kit may be provided comprising end effector 170, flexible member 190, cannula 110, actuation shaft 120, and handle 130 that are capable of being assembled by a user to form surgical instrument 100 and then later disassembled by the user resulting in the original, separate components of the kit. Further, in various embodiments, the kit may be used to assemble at least a portion of surgical instrument 100 in vivo, such that at least a portion of surgical instrument 100 is inside a patient during and after the assembly process. Also, in various embodiments, at least a portion of the surgical instrument 100 can be disassembled in vivo to facilitate the extraction of the surgical instrument from the patient.

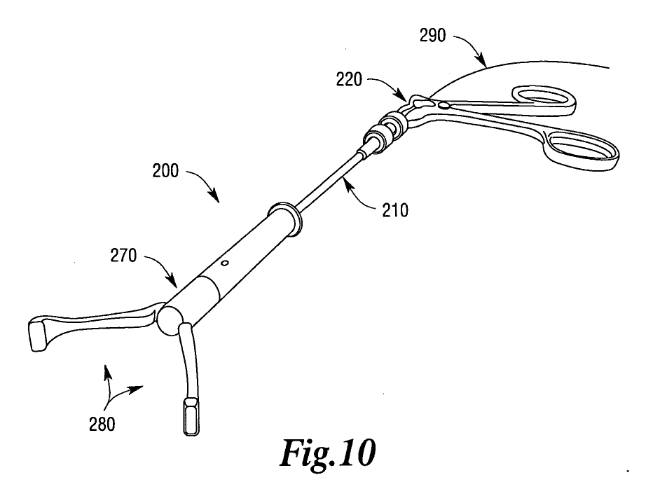

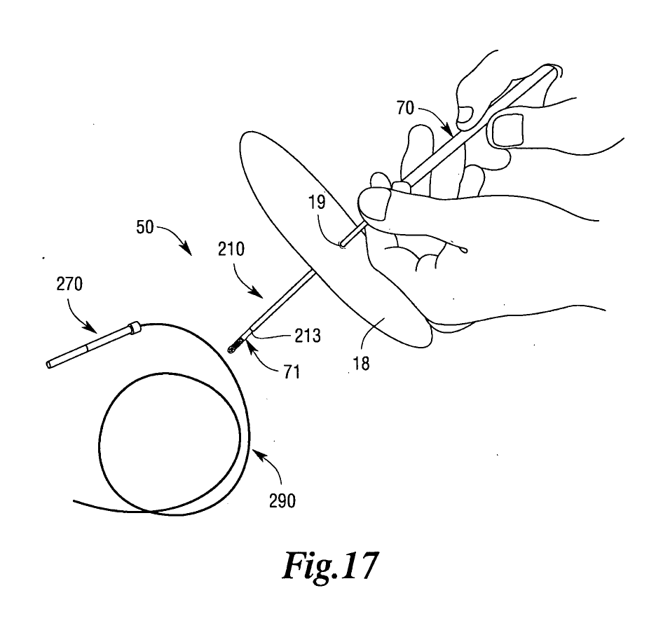

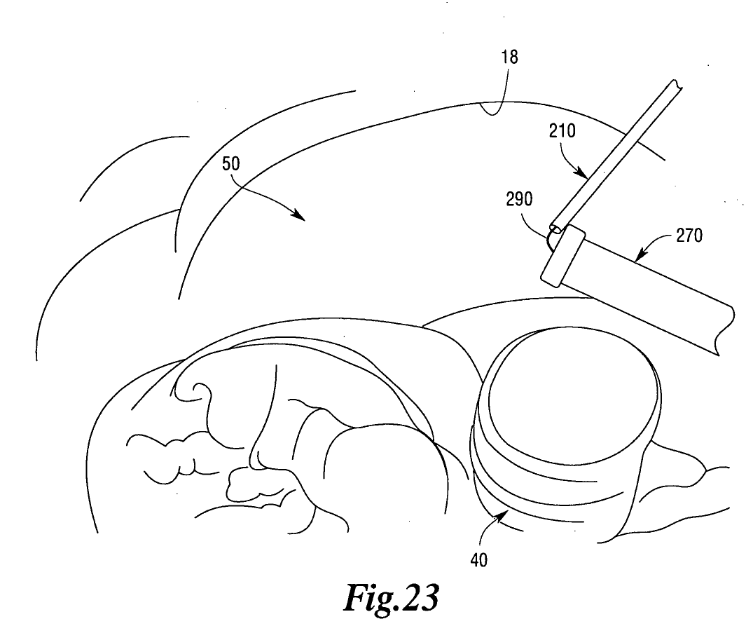

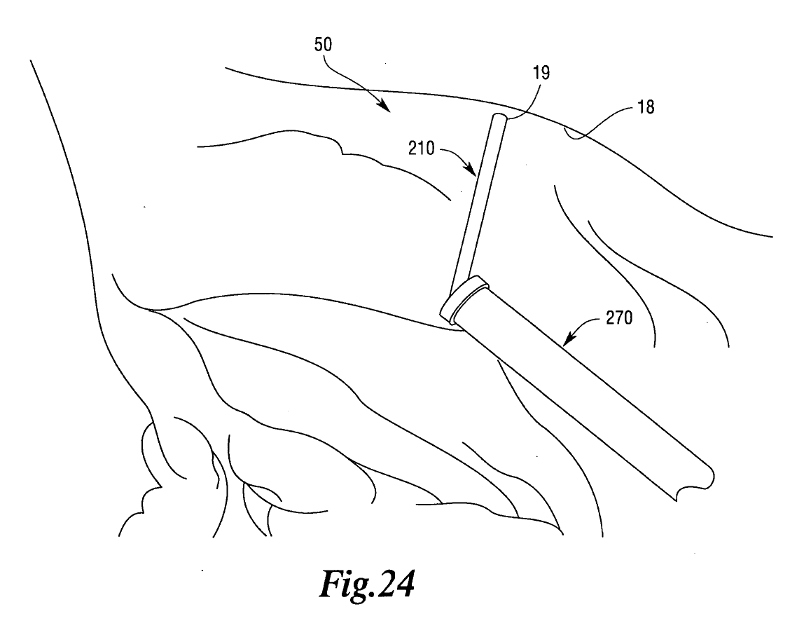

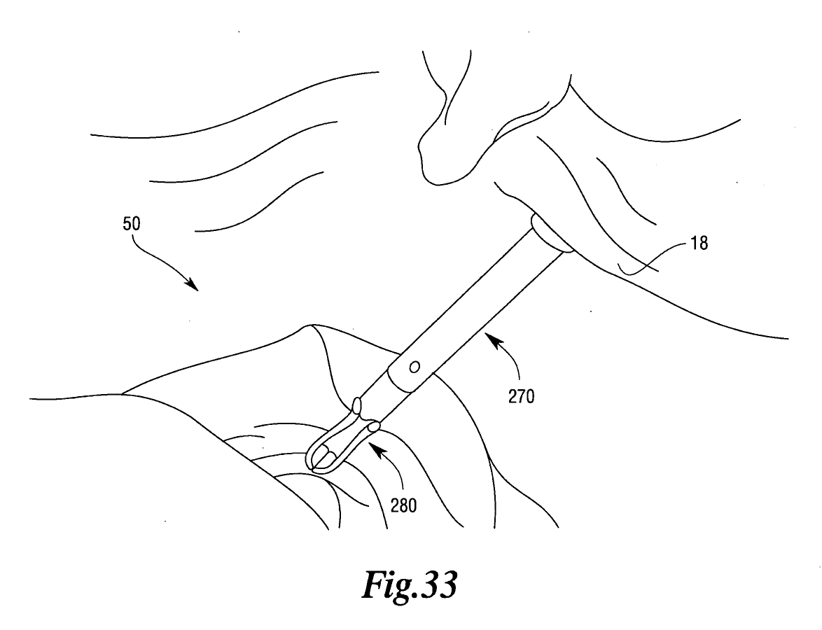

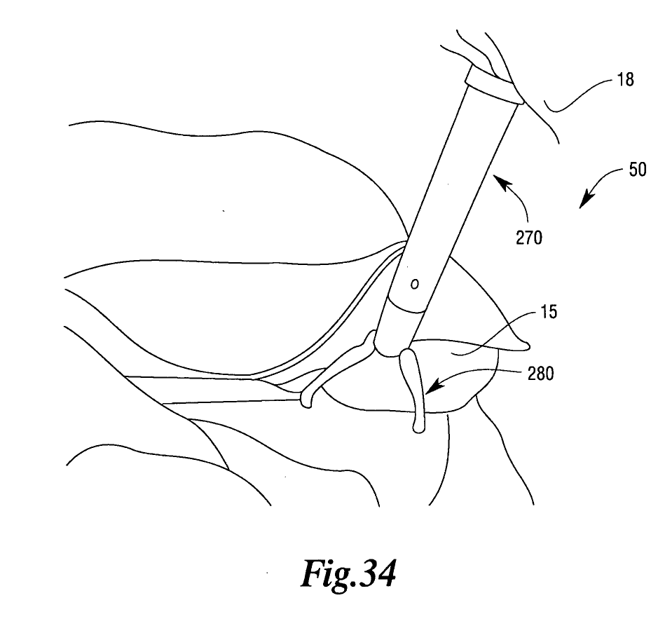

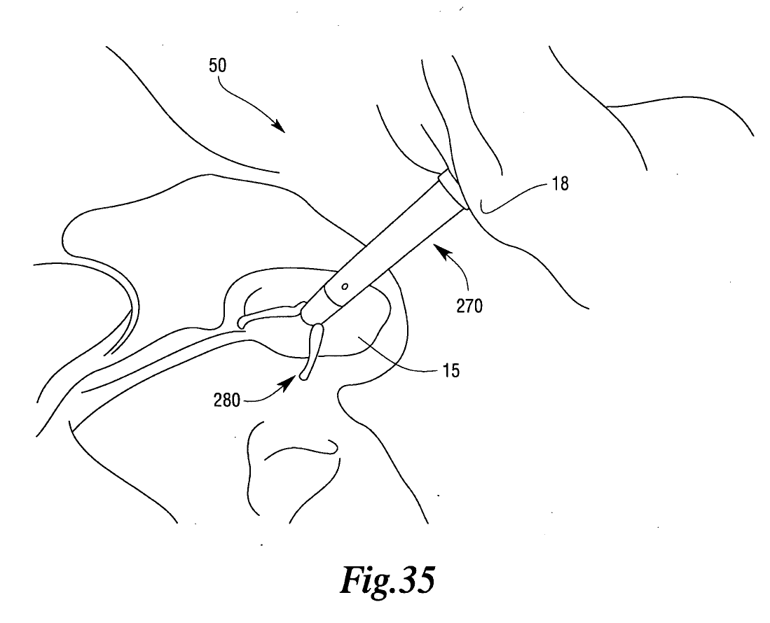

In various embodiments, referring now to FIGS. 10-35, a surgical instrument, such as surgical instrument 200, for example, may be at least partially assembled in vivo. Surgical instrument 200 may be assembled from a kit comprising, referring to FIG. 10, end effector 270, flexible member 290 extending from end effector 270, cannula 210, actuation shaft 220, and handle 230. In many respects, surgical instrument 200 is generally similar to surgical instrument 100 described above with the notable exception that actuation shaft 220 does not provide a mechanism for rotating end effector 270 about a longitudinal axis, for example.

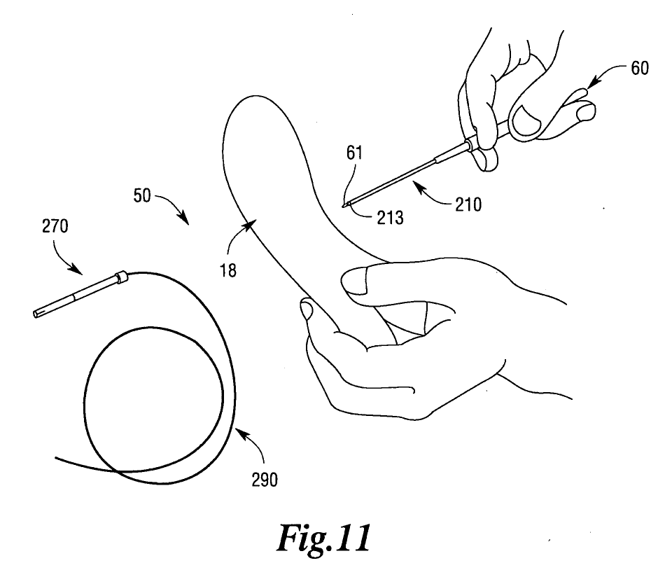

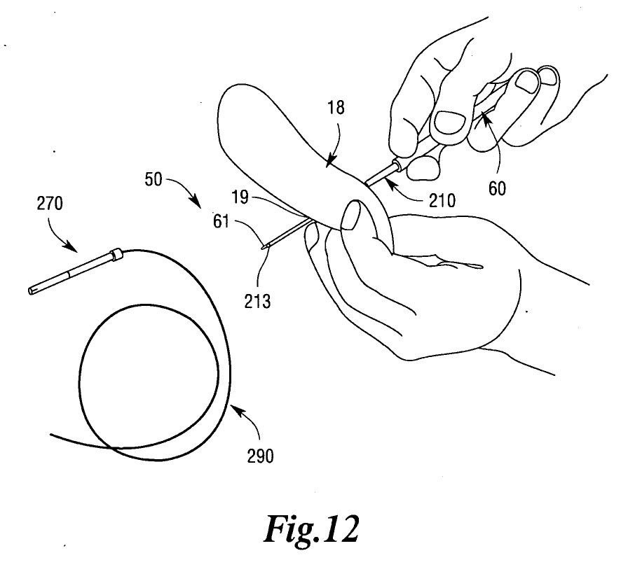

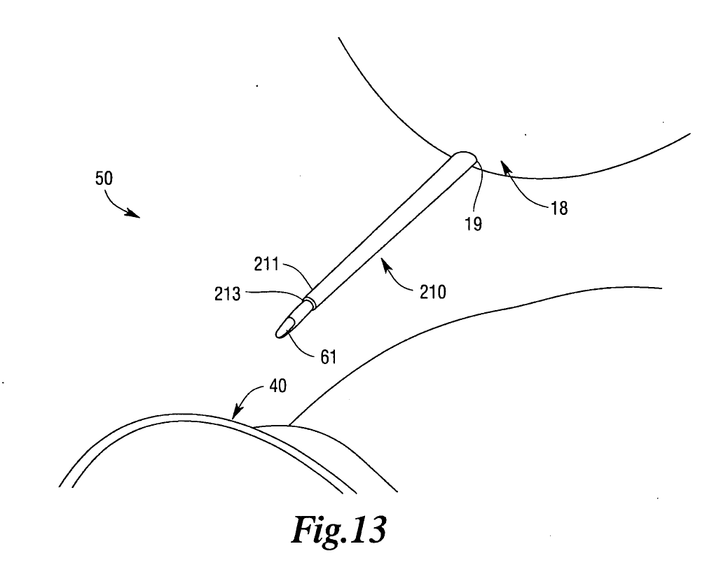

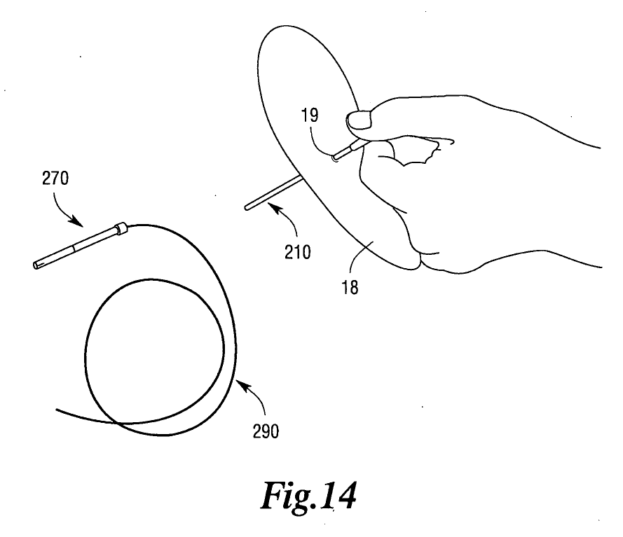

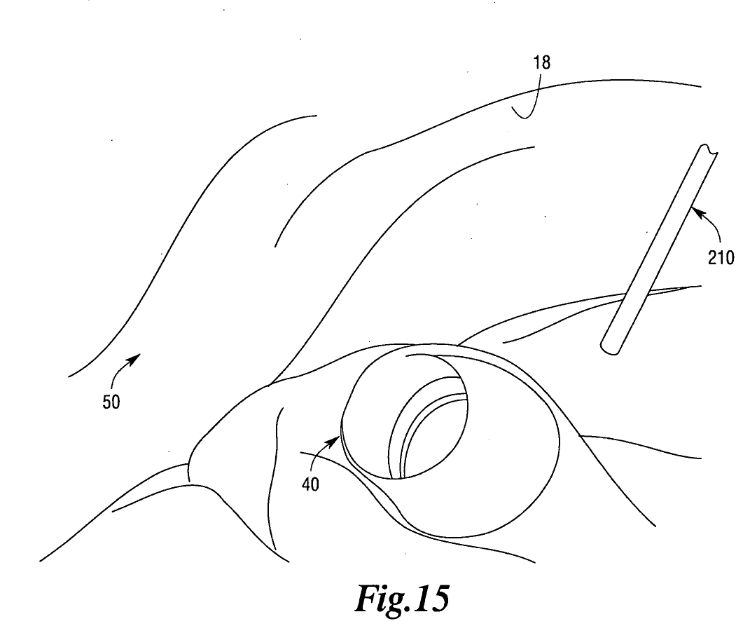

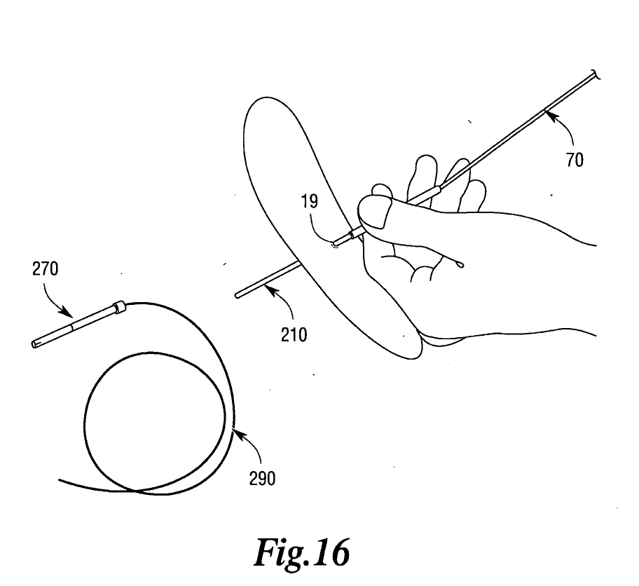

In vivo assembly of surgical instrument 200 may occur as follows. First, as described above, an overtube 40 may be introduced into a body cavity 50 of a patient, referring to FIG. 1A, wherein the body cavity may be insufflated such as by passing carbon dioxide gas through the overtube 40 and into the body cavity 50. In various circumstances, the body cavity 50 can be at least partially defined by a body wall 18 which may include the patient's abdominal wall, for example. Second, body wall 18 can be punctured to create a port, or incision 19, into body cavity 50 through body wall 18. Referring to FIGS. 11-13, body wall 18 may be punctured by inserting a puncturing device, such as Veress needle 60, for example, through an aperture 213 of cannula 210 such that a sharp tip 61 of Veress needle 60 extends from a distal end of cannula 210, and, in addition, pressing a sharp tip 61 of Veress needle 60 against and through body wall 18 to form incision 19 such that the Veress needle tip 61 and part of cannula 210 extend through body wall 18 and into body cavity 50. As the reader will appreciate, an analogue of a body cavity wall is illustrated in FIGS. 11 and 12, among others, to facilitate the reader's understanding of the steps described herein. Referring to FIG. 11, for example, the reader will note that a hand is illustrated as being positioned on the inside of the body wall analogue 18, although the reader will understand that, typically, a surgeon's hand will not be positioned on the inside of the body wall 18 of a patient (see, e.g., FIG. 13). In any event, referring to FIGS. 14 and 15, the Veress needle 60 can be removed from body cavity 50 and from cannula 210 after it has incised the body wall. Fourth, referring to FIGS. 16-18, a capturing device, suture as grasper 70, for example, can be inserted through aperture 213 of cannula 210 and into body cavity 50 in order to capture flexible member 290 as described in greater detail further below. Alternatively, the suture grasper 70 and the Veress needle 60 may be combined into a single device, thereby obviating the need for two separate devices to be inserted and removed. An exemplary combination device is provided in U.S. patent application Ser. No. 08/074,321 to Failla et al., entitled PERCUTANEOUS SUTURE EXTERNALIZER, the disclosure of which is hereby incorporated by reference in its entirety.

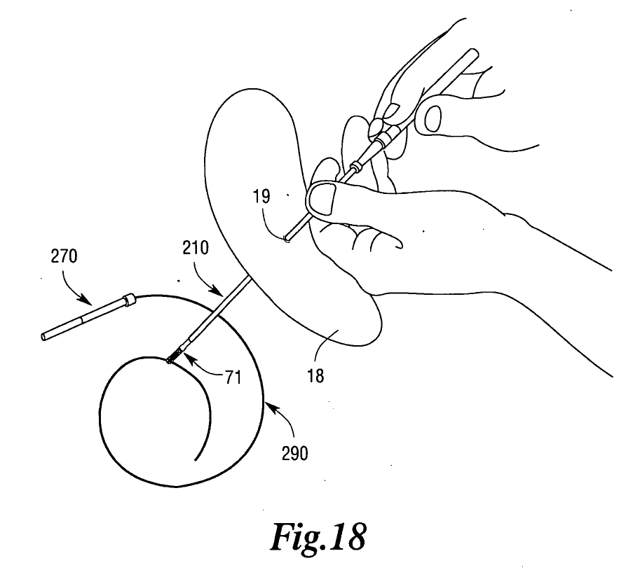

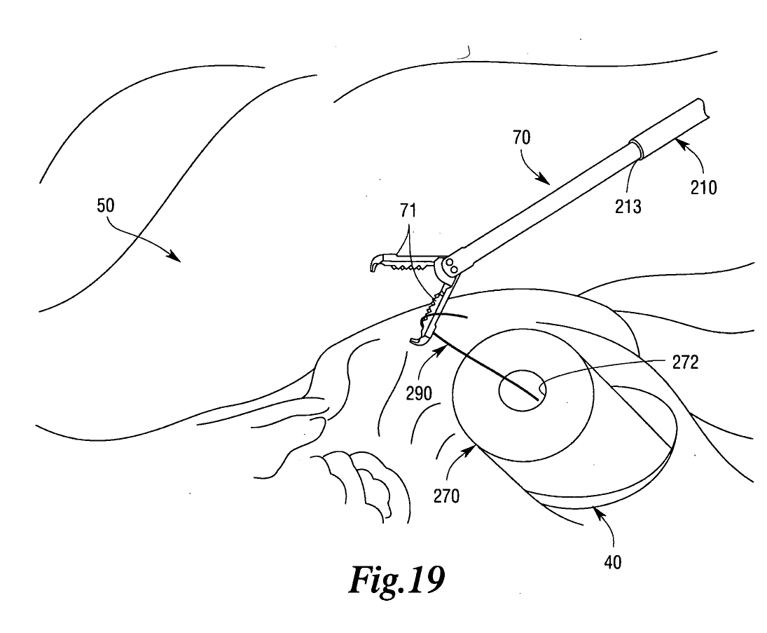

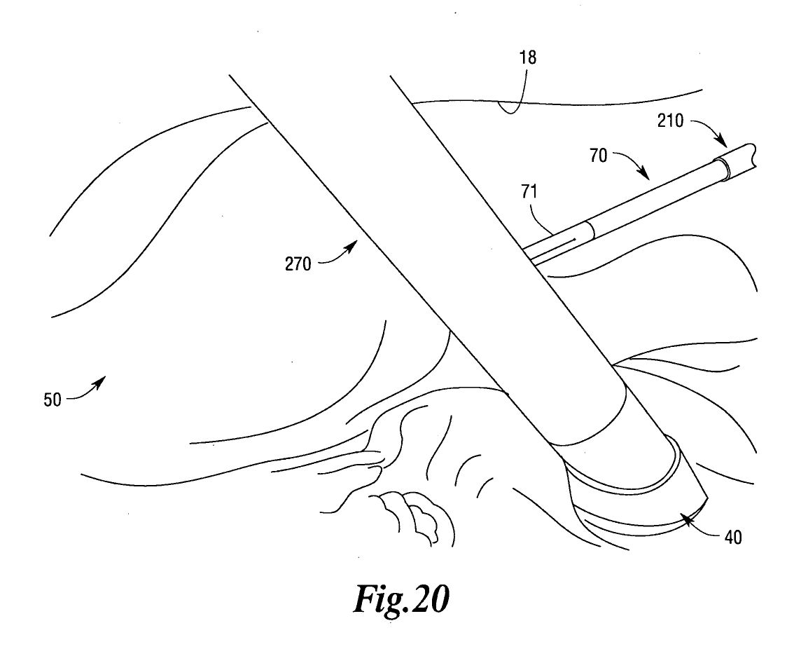

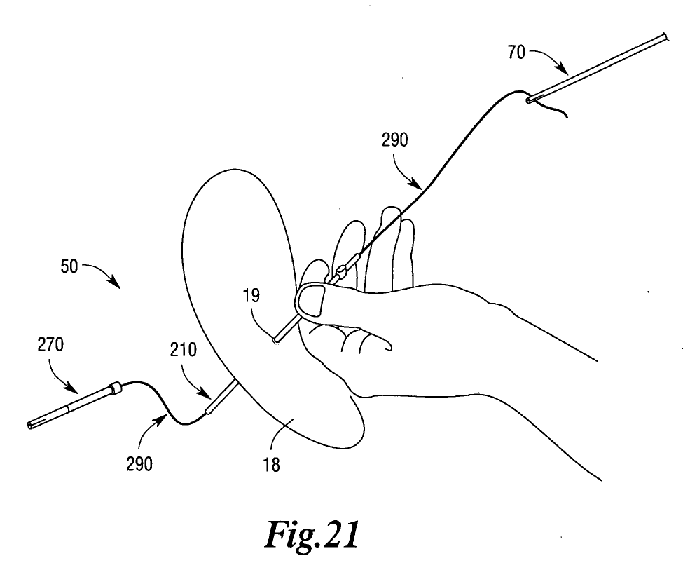

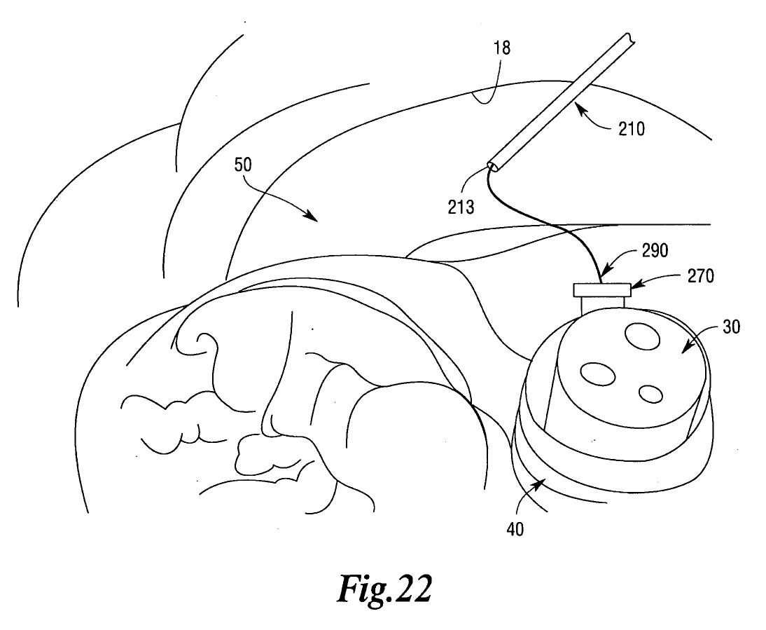

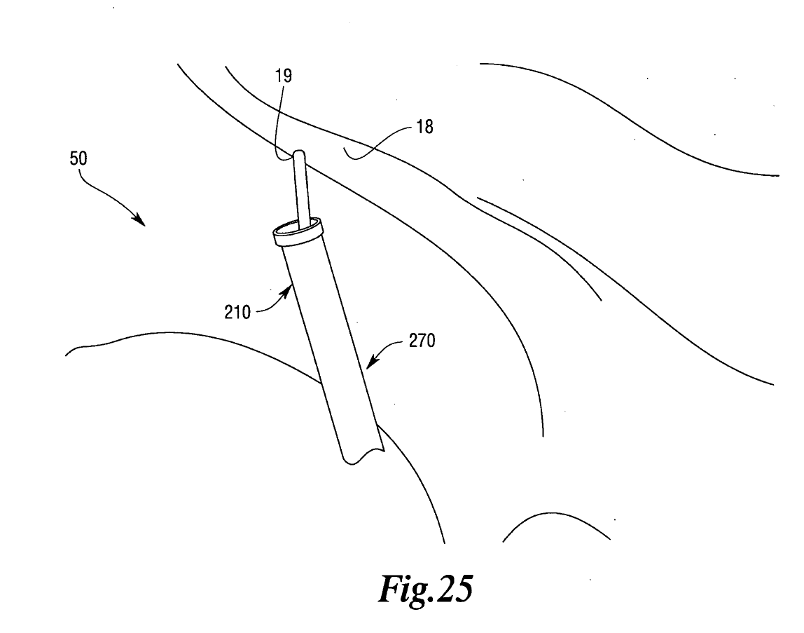

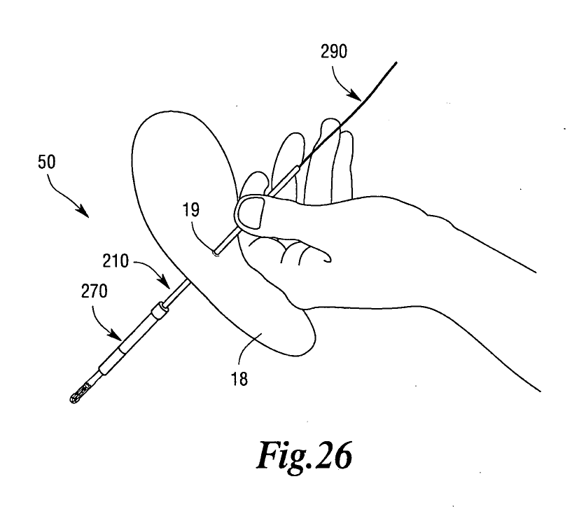

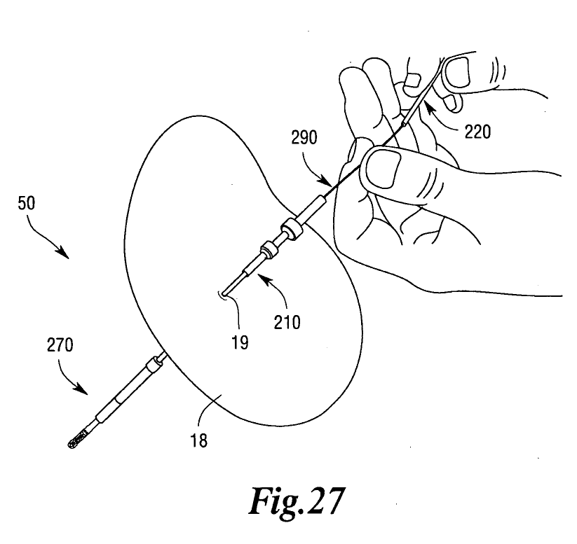

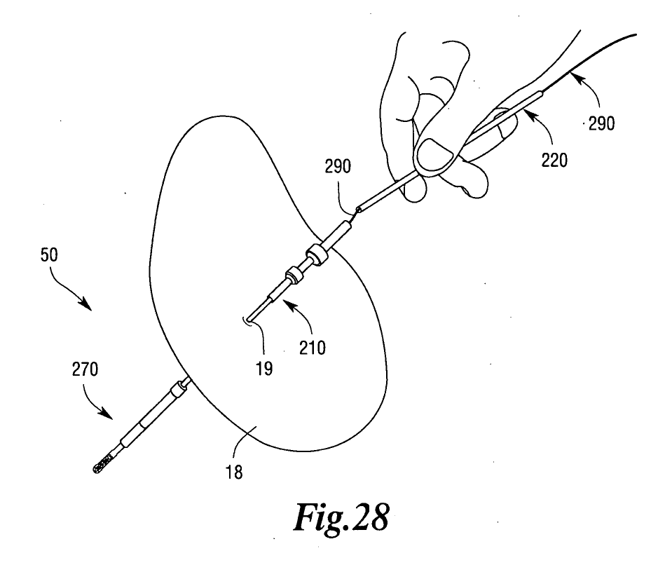

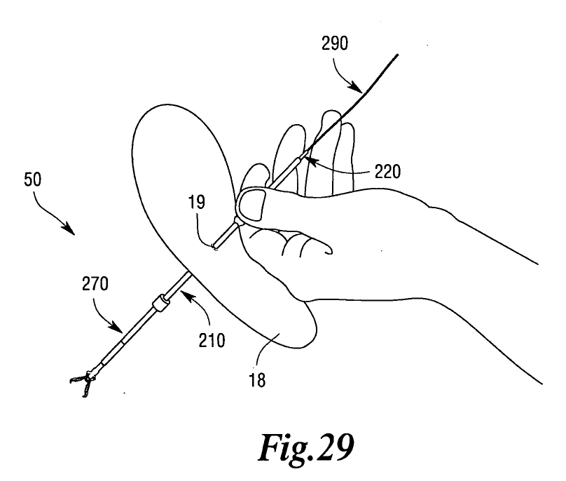

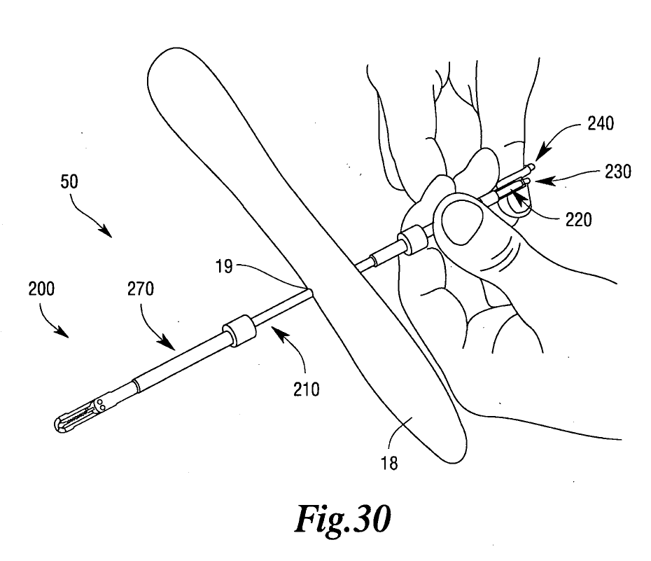

Fifth, referring to FIG. 19, the end effector 270 can be at least partially delivered to body cavity 50 through overtube 40. The end effector 270 may be passed through the overtube 40 such that flexible member 290, which is operably engaged with end effector 270 through receiving orifice 272, is oriented to enter body cavity 50 ahead or contemporaneously with end effector 270. In other embodiments, the end effector 270 can enter the body cavity ahead of the flexible member 290. In any event, endoscope 30, referring to FIG. 22, may be used to push end effector 270 through overtube 40 and into body cavity 50. Once the flexible member 290 is at least partially positioned within the body cavity, referring to FIGS. 18 and 19, grasping arms 71 of suture grasper 70 may be used to grab flexible member 290. Seventh, referring to FIGS. 20 and 21, the suture grasper 70 can be pulled out of the body cavity 50 through cannula 210 such that flexible member 290 now passes out of body cavity 50 through aperture 213 of cannula 210 and thus through incision 19. Eighth, the end effector 270 can be completely delivered to body cavity 50 by advancing endoscope 30 and/or pulling flexible member 290 further through cannula 210 (FIG. 20). Ninth, referring to FIGS. 22 and 24, the flexible member 290 can be pulled from outside the patient, such that the end effector 270 can move toward cannula 210 and such that end effector 270 and cannula 210 can begin to orient and/or align with each other's longitudinal axes. The end effector 270 may also be pulled such that it moves toward body wall 18. Tenth, referring to FIGS. 25 and 26, flexible member 290 may be further pulled from outside the patient to connect the cannula 210 to the end effector 270 inside the body cavity 50. Eleventh, referring to FIGS. 27 and 28, the flexible member 290, now extending through cannula 290 and outside the patient, may be inserted and passed through a lumen of actuation shaft 220. Twelfth, referring to FIGS. 28 and 29, the actuation shaft 220 can be translated along the flexible member 290, through aperture 213 of cannula 210, and into end effector 270. A proximal end of actuation shaft 220 can be rotated to connect the actuation shaft 220 to the end effector inside the body cavity 50 (FIG. 29). Such rotation may couple the actuation shaft 220 to an actuation member, or actuator, as described above. Thirteenth, referring to FIG. 30, a handle 230 can be connected to the cannula 210 and/or actuation shaft 220 such that actuation shaft 220 is coupled to trigger assembly 240, thereby forming surgical instrument 200. While the above provided order of steps can be utilized, various other steps can be inserted between the enumerated steps and/or the order of the steps can be rearranged as appropriate.

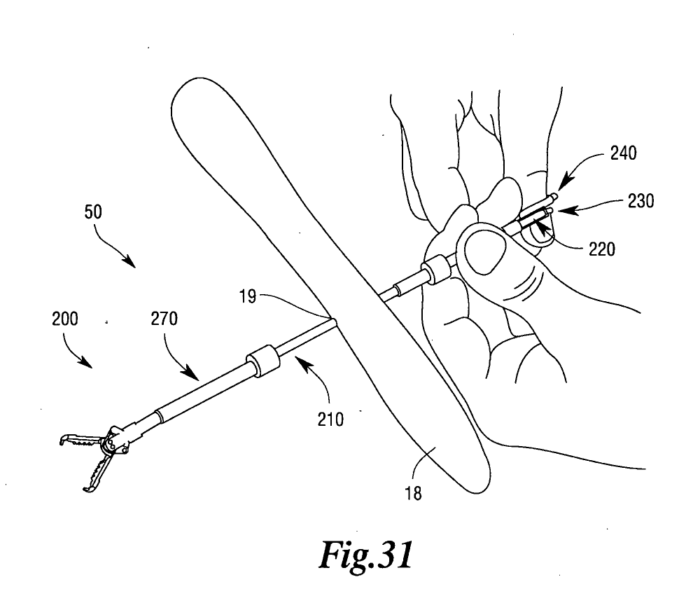

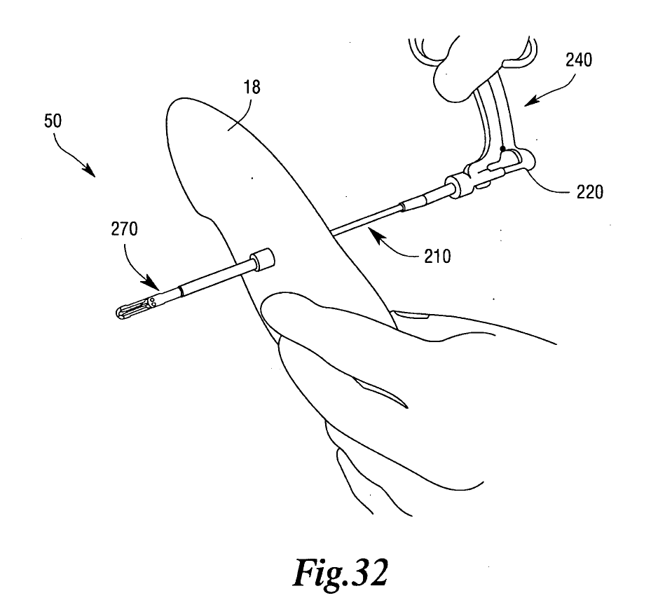

The surgical instrument 200, once assembled in vivo, may be utilized as follows. The movement or articulation of trigger assembly 240 (FIG. 31) can cause actuation shaft 220 to move relative to cannula 210. The movement of actuation shaft 220 can move an actuator of end effector 270 such that the tissue contacting portion 280 of end effector 270 is actuated (FIGS. 30, 32, and 33). Pulling the handle 230 in a proximal direction can result in cannula 210 translating proximally through incision 19; however, because end effector 270 is larger than incision 19, end effector 270 may be prevented from passing through body wall 18 (FIG. 32). In such embodiments, surgical instrument 200 may allow a user to operate with a typical laparoscopic-sized end effector through a incision or port that is much smaller (e.g. less than about 3 mm in diameter) than the end effector's diameter.

In various embodiments, as discussed above and referring to FIG. 1A, an end effector may be delivered to a body cavity within a patient through an overtube extending through a natural opening of the patient (e.g., the patient's mouth 11 and/or esophagus 12). In other various embodiments, however, an end effector may be delivered to a body cavity by any suitable delivery mode, such as through a trocar inserted through an incision in a body wall of the patient. In at least one such embodiment, a surgeon may make an incision in the patient and insert a trocar through the incision such that the end effector can be passed through an aperture in the trocar and into the body cavity. In certain circumstances, a surgeon may make a first incision in the patient to insert the end effector 170 into a body cavity through a trocar and, in addition, a second incision in order to insert the cannula 110 into the body cavity, wherein the end effector 170 can be assembled to the cannula 110 in vivo. In other circumstances, a surgeon may make an incision and insert both the end effector 170 and the cannula 110 through the same incision such that the end effector 170 and the cannula 110 can be assembled in vivo. In such circumstances, the surgeon can insert a trocar into the incision which has an aperture large enough to receive the end effector 170 and the cannula 110.

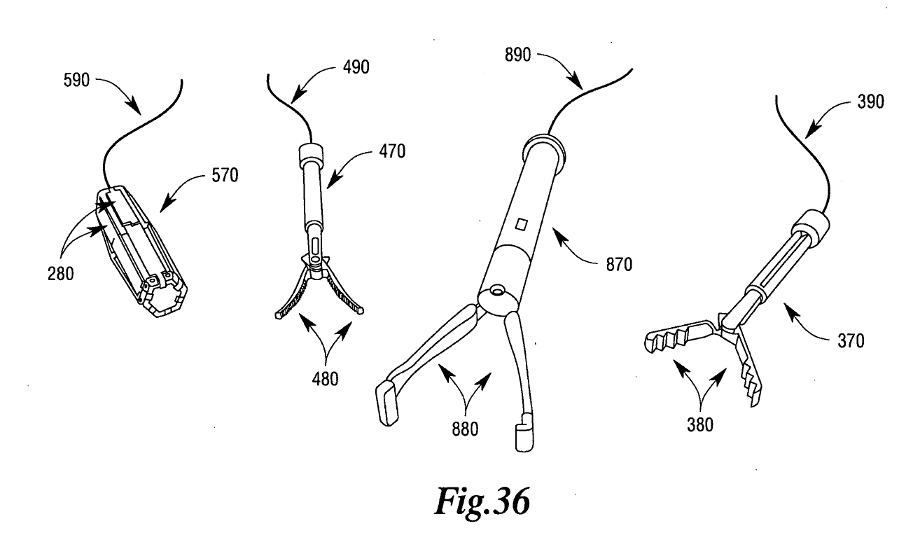

In various embodiments, different end effectors may be used in conjunction with a surgical kit to assemble a surgical instrument in vivo, as described above. For example and with reference to FIG. 36, such end effectors may include, but are not limited to, an expandable bolster 570, a 5 mm Maryland-style dissector 470, a 10 mm Babcock-style grasper 870, and/or a 5 mm standard grasper 370. Each end effector 570, 470, 870, 370 may include a tissue contacting portion 580, 480, 880, 380, respectively, and a flexible member 590, 490, 890, 390, respectively, extending from the respective end effector, 570, 470, 870, 370. Additionally, the end effectors described in U.S. patent application Ser. No. 11/693,976 to Coe et al., entitled DETACHABLE END EFFECTORS, the disclosure of which is hereby incorporated by reference in its entirety, are also adaptable to be likewise used or included in such a surgical kit. Other exemplary end effectors can include, but are not limited to, a specimen retrieval bag, biopsy jaws with a spike, a snare loop, scissors, and/or a hook knife, for example. Various end effectors are described in greater detail in commonly-owned U.S. patent application Ser. No. 12/133,109 to Zwolinski et al., entitled ENDOSCOPIC DROP OFF BAG; U.S. patent application Ser. No. 11/610,803 to Nobis et al., entitled MANUALLY ARTICULATING DEVICES; and U.S. patent application Ser. No. 12/133,953 to Nobis et al., entitled MANUALLY ARTICULATING DEVICES, the disclosures of which are incorporated by reference in their entirety. Any of these end effectors and/or any other suitable end effectors may be used as part of a surgical kit comprising a cannula, such as cannula 110, for example, an actuation shaft, such as actuation shaft 120, for example, and/or a handle, such as handle 130, for example.

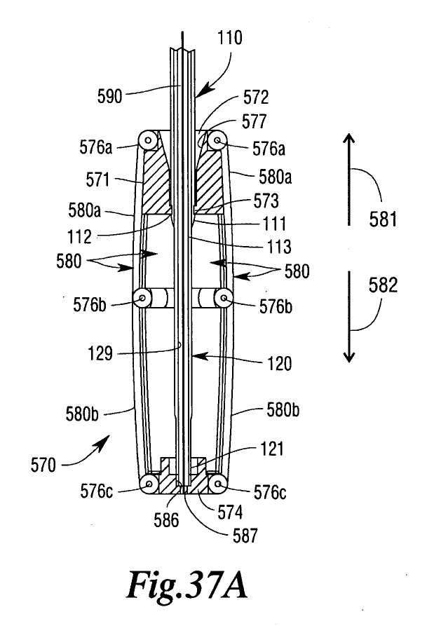

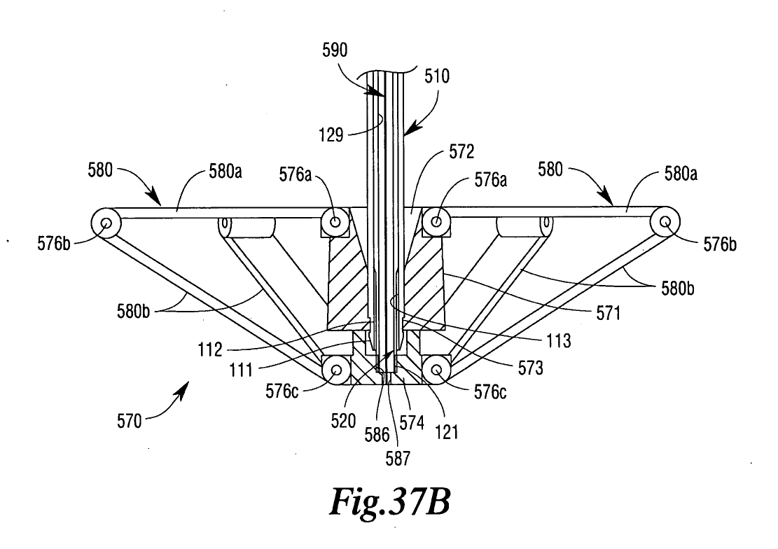

Referring now to FIGS. 36-38B, in various embodiments, expandable bolster 570 can comprise a housing 571, a receiving orifice 572, an actuator 574, and a tissue contacting portion 580 operably coupled to the housing 571 and to the actuator 574 (see FIG. 37A). The receiving orifice 572 comprises a chamfered surface 577 which, as described above with respect to end effector 170, is sized and configured to assist in positioning and locating cannula 110 in receiving orifice 572. Receiving orifice 572 may also include a protrusion 573 that is configured to be engaged by cannula recess 112 when the connector portion 111 of cannula 110 is inserted into the receiving orifice 572. In at least one embodiment, receiving orifice 572 can be made from a resilient, elastic material such that protrusion 573 may be resiliently engaged with, or snapped into, recess 112 when an appropriate amount of force is applied to the cannula 110 and/or to the end effector 570, thereby forming a secure, but releasable connection between the connector portion 111 and the expandable bolster 570. Further to the above, flexible member 590 may extend from the end effector 570, see FIG. 36, wherein the flexible member 590 may be attached to the end effector 570 and extend out of the end effector 570 through receiving orifice 572, as shown in FIG. 37A. In certain embodiments, flexible member 590 may be attached to actuator 574 of end effector 170 by gluing, welding, or knotting flexible member 590 in a hole 587 in actuator 574, for example. As described above with respect to flexible member 190, flexible member 590 may take the form of a wire, cable, and/or cord, for example. Additionally, flexible member 590 may extend through actuator 574 and form a loop (not shown) through which end effector 570 may be retrieved from a body cavity with graspers and the like, as described above and referenced below.

As indicated above, end effector 570 may include at least one tissue contacting portion 580 extending from the housing 571 of the end effector 570. The tissue contacting portion 580 may comprise proximal arms 580a and distal arms 580b pivotably connected to each other by intermediate pins 576b. The proximal and distal arms 580a and 580b may also be pivotally coupled to housing 571 by proximal pins 576a and to actuator 574 by distal pins 576c. Accordingly, both proximal and distal arms 580a and 580b are operably connected to actuator 574. As the reader will appreciate, as described in greater detail below, the actuator 574 can be moved in order to expand or deploy proximal arms 580a and distal arms 580b. In various embodiments, actuator 574 may further comprise a threaded portion 586, wherein the threaded portion 586 can comprise threads which can be configured to be mateably engage actuation shaft 120 as described above with respect to end effector 170.

In various embodiments, the actuator 574 may be moved between first and second positions in the directions indicated by arrows 581 and 582 (FIG. 37A), for example. When the actuator 574 is moved in the direction indicated by arrow 581, i.e., toward housing 571, the proximal and distal arms 580a and 580b can toggle open to form an expanded, actuated configuration of end effector 570, as seen in FIG. 37B. When the actuator 574 is moved in the direction indicated by arrow 582, i.e., away from housing 571, the proximal and distal arms 580a and 580b can toggle closed to form a collapsed, unactuated configuration of end effector 570, as seen in FIG. 37A. Accordingly, the proximal and distal arms 580a and 580b can cooperate with one another and act in a similar manner as a toggle bolt in order to assume either a collapsed, unactuated configuration or an expanded, actuated configuration.

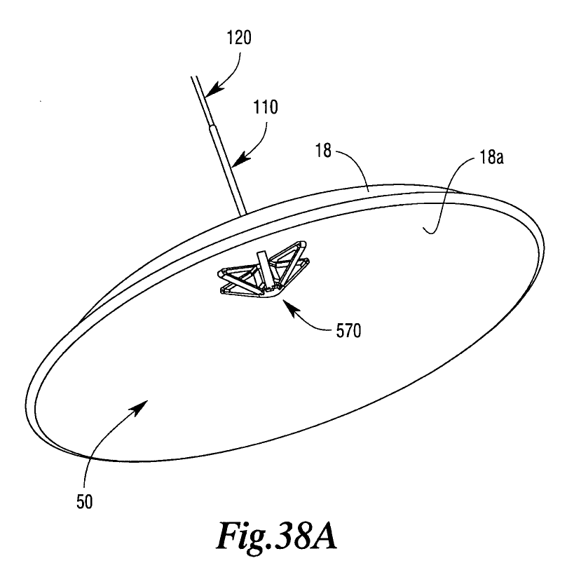

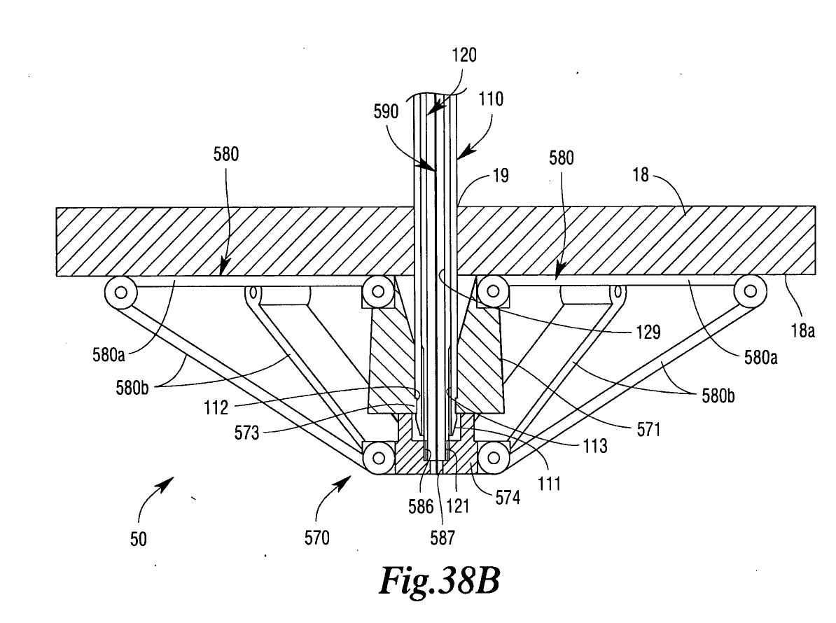

In use, a surgical kit comprising expandable bolster 570, flexible member 590 extending from expandable bolster 570, cannula 110, actuation shaft 120, and handle 130 may be at least partially assembled in vivo in order to form a surgical instrument in a similar fashion to that described above in connection with end effectors 170 and 270. Referring to FIGS. 1A and 38A-38B, expandable bolster 570 may be delivered to a body cavity 50 of a patient through a first opening, such as the mouth of the patient, for example, the cannula 110 can be at least partially inserted into the body cavity 50 through a second opening, such as incision 19 of body wall 18, for example, the expandable bolster 570 can be assembled to the cannula 110, and the remainder of the surgical instrument can be assembled thereto in order to form a surgical instrument. Thereafter, the expandable bolster 570 may be actuated by articulating trigger assembly 140 (see FIGS. 6A-6D) such that actuation shaft 120 and actuator 574 move in a proximal direction, i.e., in the direction of arrow 581 (FIG. 37A). After bolster 570 has been actuated to an expanded configuration, the expandable bolster 570 may be pulled in a proximal direction, i.e., in the direction of arrow 581, by handle 130, for example, such that tissue contacting portion 580, including proximal arms 580a, for example, can abut and/or press against an inner surface 18a of body wall 18, see FIG. 38B. The continued pulling of handle 130 can retract body wall 18 and create a working space, or at least a larger working space, within body cavity 50, see FIG. 38A. Such a working space may be helpful in performing a surgical procedure where another surgical tool is introduced into body cavity 50, for example. Such a surgical tool may include another surgical instrument assembled in vivo, an endoscopic tool introduced through a working channel port 38 of an endoscope 30 (see, e.g., FIGS. 1A and 2), or a traditional laparoscopic tool inserted through a trocar, for example. While the above provided order of steps can be utilized, various other steps can be inserted between the enumerated steps and/or the order of the steps can be rearranged as appropriate.

Disassembly of a surgical instrument utilizing end effector 570 may occur in a similar manner as the manner used to disassemble surgical instrument 100 described above. First, the expandable bolster can be returned to a collapsed, closed configuration (FIG. 37A). Second, the actuation shaft 120 can be disengaged from threaded portion 586 of actuator 574. Third, the actuation shaft 120 can be used to push the expandable bolster 570 off of the actuation shaft 120. Alternatively, the cannula 110 can be used to pull expandable bolster against body wall 18 such that end effector 570 can decouple from cannula 110. In any event, once the cannula 110 has been detached from end effector 570, the cannula 110 can be removed from the body cavity 50 through incision 19 in body wall 18 and the expandable bolster 570 and flexible member 590 can be removed from the body cavity 50 through overtube 40 (FIG. 1A) as described above with respect to end effector 170. Note that the internal friction between proximal and distal arms 580a and 580b, housing 571, and actuator 574 may be sufficient to keep the expandable bolster 570 in a collapsed configuration while retrieving the bolster 570 through overtube 40. Alternatively, in at least one embodiment, a Nitinol (nickel titanium) wire, for example, may be incorporated into the proximal pins 576a, intermediate pins 576b, and/or distal pins 576c such that the proximal arms 580a and/or distal arms 580b can be biased toward the collapsed, closed position shown in FIG. 37A. While the above provided order of steps can be utilized, various other steps can be inserted between the enumerated steps and/or the order of the steps can be rearranged as appropriate.

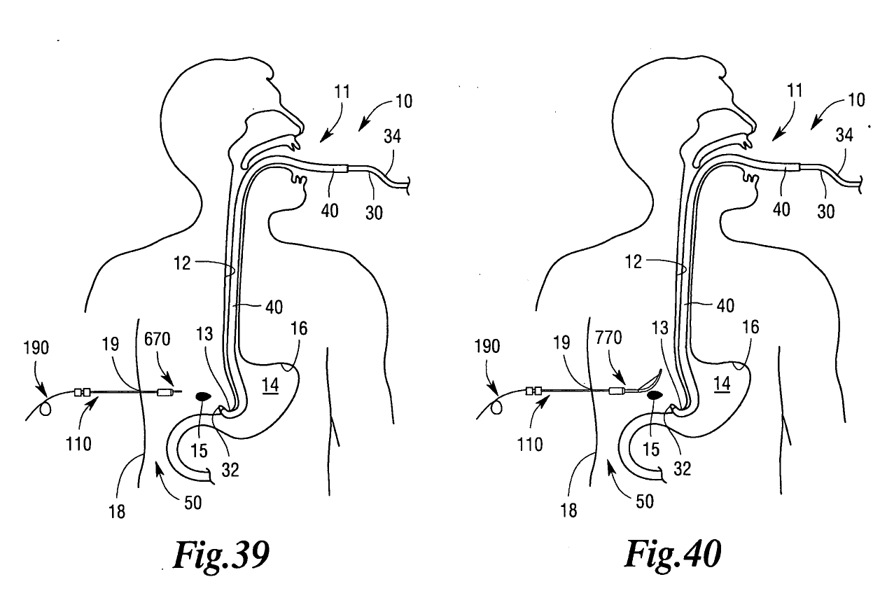

While various embodiments described above include actuatable end effectors, or end effectors including a relatively movable tissue contacting portion, non-actuatable end effectors, such as those including relatively static, or stationary, tissue contacting portions, for example, are also contemplated. The terms static and stationary do not mean that the end effector cannot be moved at all; rather, a static or stationary end effector is one that can be moved within a surgical site, for example, but is not moved relative to the rest of the surgical instrument, once assembled thereto. In various embodiments, a stationary portion of an end effector can comprise a knife blade, for example. In certain embodiments, referring now to FIGS. 39-40, an end effector may include a needle knife 670 (FIG. 39) and/or a sphincterotome 770 (FIG. 40). In various embodiments, the needle knife 670 can comprise an elongate wire for cutting and/or coagulating tissue and the sphincterotome 770 can comprise a bow-type wire configuration for cutting and/or coagulating tissue. In certain embodiments, the needle knife 670 and sphincterotome 770 can also be configured to receive electrical current, or energy, which can facilitate the cutting and/or coagulation of tissue. In use, in various embodiments, a static end effector, such as end effectors 670 and 770, for example, can be placed in a body cavity of a patient utilizing any suitable technique, such as those disclosed in this application, for example, wherein a cannula, such as cannula 110, for example, can be connected to the static surgical instrument in vivo utilizing any suitable technique, such as those disclosed in this application, for example. In at least one such embodiment, the end effector 670 and/or end effector 770 can further comprise a flexible member, such as flexible member 190, for example, connected thereto, wherein the flexible member 190 can be pulled through an aperture in cannula 110 in order to engage the end effectors 670 or 770 with the cannula 110. In various embodiments, further to the above, the end effectors can be press-fit or snap-fit onto a cannula 110. In various embodiments, a cannula and an end effector can comprise electrical contacts which can be engaged with one another when the end effector is attached to the cannula. For example, the cannula can comprise a first conductor having a first electrical contact and a second conductor having a second electrical contact and, in addition, the end effector can comprise first and second electrical contacts which can be configured to engage the first and second electrical contacts of the cannula, respectively. Once the first electrical contact of the cannula is engaged with the first electrical contact of the end effector and, similarly, the second electrical contact of cannula is engaged with the second electrical contact of the end effector, electrical current from a power source can flow through the first conductor, the end effector, and the second conductor in order to supply the end effector with electrical current as indicated above. For example, the current flowing through the end effector can flow through the wires of the needle knife tip 670 or sphincterotome 770. In at least one embodiment, the first electrical contacts can be surrounded by an insulative material and the second electrical contacts can surround the insulative material such that current does not flow between the first and second contacts. In at least one such embodiment, the first and second electrical contacts can comprise concentric or annular configurations which can permit the contacts to be operably connected regardless of the rotational alignment between the end effector and the cannula, for example. In any event, once assembled, in various embodiments, a surgeon may control the needle knife 670 or sphincterotome 770 with the cannula 110. In various embodiments, a handle may also be attached to the cannula 110 so that a user may have better control over the movement of needle knife 670 or sphincterotome 770 inside the body cavity. In at least one such embodiment, the handle can comprise a switch which, when actuated, can be configured to allow current to flow to the end effector as outlined above. Any of these end effectors and/or any other suitable end effectors may be part of a surgical kit comprising a cannula, such as cannula 110, for example, and/or a handle, such as handle 130, for example.

The devices disclosed herein can be designed to be disposed of after a single use, or they can be designed to be used multiple times. In either case, however, a device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present disclosure and appended claims.

Preferably, the various embodiments described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide, and/or steam.

Although various embodiments have been described herein, many modifications and variations to those embodiments may be implemented. For example, the surgical instrument may be assembled in vivo without ultimately including a handle. In such embodiments, a user may actuate the end effector by manually moving the actuation shaft relative to the cannula. Additionally, the actuation shaft may be unitary and integral with the handle and/or the cannula may be unitary and integral with the handle. Additionally, while the above shows a puncturing device and a grasping device being inserted through the body wall from outside the patient, the body wall may alternatively be pierced from inside the body cavity by use of an appropriate puncturing device that is passed into the body cavity via an endoscope/overtube as shown in FIG. 1A. In such embodiments, the flexible member may also be passed out of the body cavity and through a body wall by use of an endoscopic tool through an overtube. Also, where materials are disclosed for certain components, other materials may be used. The foregoing description and following claims are intended to convey and cover all such modification and variations.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference.

What is claimed is:

1. A surgical kit, comprising:
an end effector configured to be delivered into a body cavity of a patient;
a flexible member extending from said end effector; and
a cannula comprising:
a first end configured to be inserted into the body cavity, said first end comprising a connector portion configured to be releasably attached to said end effector;
a second end; and
an aperture, wherein said aperture is sized and configured to receive at least a portion of said flexible member.

2. The surgical kit of claim 1, further comprising an actuation shaft comprising an attachment portion, wherein said attachment portion is configured to be releasably attached to said end effector such that operation of said actuation shaft can operate said end effector.

3. The surgical kit of claim 2, wherein said actuation shaft is configured to be inserted into said aperture of said cannula, wherein said actuation shaft further comprises a second aperture, and wherein said second aperture is sized and configured to receive at least a portion of said flexible member.

4. The surgical kit of claim 2, wherein said end effector further comprises a tissue contacting portion, and wherein said actuation shaft is configured to move said tissue contacting portion.

5. The surgical kit of claim 1, wherein said end effector comprises an expandable bolster.

6. A surgical instrument, comprising:
an end effector configured to be delivered into a body cavity of a patient through a natural opening in the patient;
an elongate flexible member connected to said end effector;
a cannula comprising:
a first end configured to be inserted into the body cavity through a second opening in the patient, said first end comprising a connector portion configured to be releasably attached to said end effector;
a second end; and
an aperture, wherein said aperture is sized and configured to receive at least a portion of said flexible member; and
an actuation shaft comprising an attachment portion, wherein said attachment portion is configured to be releasably attached to said end effector such that operation of said actuation shaft can operate said end effector.

7. The surgical instrument of claim 6, wherein said actuation shaft is configured to be inserted into said aperture of said cannula, wherein said actuation shaft further comprises a second aperture, and wherein said second aperture is sized and configured to receive at least a portion of said flexible member.

8. The surgical instrument of claim 6, wherein said end effector further comprises a tissue contacting portion, and wherein said actuation shaft is configured to move said tissue contacting portion.

9. The surgical instrument of claim 6, wherein said end effector comprises an expandable bolster.

10. A method of assembling a surgical instrument inside a patient, said method comprising the steps of:
delivering an end effector to a body cavity of the patient, wherein said end effector is operably engaged with a flexible member;
inserting a shaft into the body cavity;
pulling said flexible member relative to said shaft such said end effector moves relative to said shaft; and
connecting said shaft to said end effector inside the body cavity.

11. The method of claim 10, wherein said delivering step comprises delivering said end effector to the body cavity through a natural opening in the patient, and wherein said inserting step comprises inserting said shaft into the body cavity through a second opening in the patient.

12. The method of claim 10, further comprising the step of translating said shaft along said flexible member.

13. The method of claim 10, wherein said shaft comprises a cannula, and wherein the method further comprises the step of passing said flexible member through said cannula after said inserting step and before said pulling step.

14. The method of claim 13, further comprising the steps of inserting said flexible member through an actuation shaft and translating said actuation shaft along said flexible member, through said cannula, and into said end effector.

15. The method of claim 14, further comprising the step of coupling said actuation shaft to an actuator of said end effector.

16. The method of claim 15, further comprising the step of attaching said actuation shaft and said cannula to a handle.

17. The method of claim 16, further comprising the step of moving said actuation shaft relative to said cannula with said handle.

18. The method of claim 10, wherein said pulling step comprises passing at least a portion of said flexible member through a body wall of the patient and further pulling said flexible member such that said end effector moves toward said body wall.

19. The method of claim 10, wherein said end effector comprises an expandable bolster.

20. The method of claim 19, further comprising the steps of expanding said expandable bolster inside the body cavity to form an expanded bolster, and pulling said expanded bolster against a body wall of the patient to retract the body wall.
